(19) United States
(12) Patent Application Publication
(10) Pub. No.: US 2021/0023094 A1
(43) Pub. Date: Jan. 28, 2021

(54) PSMA ANTIBODY-DRUG CONJUGATES

(71) Applicant: PSMA Development Company, LLC, New York, NY (US)

(72) Inventors: Dangshe Ma, Millwood, NY (US); Paul J. Maddon, Scarsdale, NY (US); William C. Olson, Yorktown Heights, NY (US); Svetlana O. Doronina, Snohomish, WA (US); Brian E. Toki, Shoreline, WA (US); Peter D. Senter, Seattle, WA (US)

(73) Assignee: PSMA Development Company, LLC, New York, NY (US)

(21) Appl. No.: 16/921,811

(22) Filed: Jul. 6, 2020

Related U.S. Application Data
(62) Division of application No. 11/471,457, filed on Jun. 20, 2006, now abandoned.
(60) Provisional application No. 60/792,360, filed on Apr. 14, 2006.
(60) Provisional application No. 60/692,399, filed on Jun. 20, 2005.

(51) Int. Cl. A61K 31/573; A61K 31/337; A61K 38/19; A61K 38/21; A61K 38/20; A61K 47/68

(52) U.S. Cl. CPC A61K 31/573; A61K 31/337; A61K 38/191; A61K 47/6811; A61K 38/2013; A61K 47/6889; A61K 47/6869; A61K 38/21

(57) ABSTRACT

This invention relates generally to antibody-drug conjugates (ADCs). In particular, the invention relates to ADCs which comprise an antibody or antigen-binding fragment thereof which binds to prostate-specific membrane antigen (PSMA) and is conjugated to monomethylauristatin norephedrine or monomethylauristatin phenylalanine. The antibody-drug conjugate has a PC-3™ cell to C4-2 or LNCaP™ cell selectivity of at least 250. The invention also relates, in part, to compositions of and methods of using the ADCs. The methods provided include, for example, methods for treating a PSMA-mediated disease.

### RELATED APPLICATIONS

This application is a divisional of U.S. application Ser. No. 11/471,457, filed Jun. 20, 2006, which claims the benefit under 35 U.S.C. § 119 of U.S. Provisional Application No. 60/692,399, filed Jun. 20, 2005 and U.S. Provisional Application No. 60/792,360, filed Apr. 14, 2006, the contents of each of which are incorporated herein by reference in their entirety.

### GOVERNMENT SUPPORT

Aspects of the invention may have been made using funding from National Institutes of Health Grants CA107653 (DM) and CA96075 (GPD). Accordingly, the government may have rights to the invention.

### FIELD OF THE INVENTION

This invention relates generally to antibody-drug conjugates (ADCs). In particular, the invention relates to ADCs which comprise an antibody or antigen-binding fragment thereof which binds to prostate-specific membrane antigen (PSMA) and is conjugated to monomethylauristatin norephedrine (MMAE) or monomethylauristatin phenylalanine (MMAF). The antibody-drug conjugate has a PC-3™ cell to C4-2 or LNCaP™ cell selectivity of at least 250. The invention also relates, in part, to compositions of and methods of using the ADCs. The methods provided include, for example, methods for treating a PSMA-mediated disease.

### BACKGROUND OF THE INVENTION

Prostate cancer is the most common malignancy and the second leading cause of cancer death in men in the United States (Jemal A, et al., CA Cancer J Clin 2005; 55:10-30). Localized prostate cancer typically is treated with surgery or radiation, and recurrent disease can be controlled temporarily with androgen ablation (Klein E A, et al., Urol Clin North Am 2003; 30:315-30). However, almost all prostate carcinomas eventually become hormone refractory and then rapidly progress (Denmeade S R, et al., Nat Rev Cancer 2002; 2:389-96). Hormone-refractory or androgen-independent prostate cancer has proven to be largely resistant to conventional chemotherapy. With the exception of palliative care, the only approved chemotherapy is docetaxel in combination with prednisone, which offers a modest (2.4 month) survival benefit (Gulley J, et al., Am J Ther. 2004; 351:1513-20; Petrylak D P, et al., New Engl J Med 2004; 351:1513-20). New molecularly targeted therapies are needed.

### SUMMARY OF THE INVENTION

The invention provided herein relates to ADCs that exhibit particularly high selectivity. In one aspect of the invention an antibody-drug conjugate is provided that comprises an antibody or antigen-binding fragment thereof which binds to PSMA and is conjugated to monomethylauristatin norephedrine or monomethylauristatin phenylalanine, wherein the antibody-drug conjugate has a PC-3™ cell to C4-2 or LNCaP™ cell selectivity of at least 250. In one embodiment, the selectivity is at least 500, 1000, 2500, 6000 or 13,000. In another embodiment, the selectivity is 1567, 6286 or 13,636. In some embodiments, the antibody or antigen-binding fragment thereof is conjugated to at least 3, 4 or 5 monomethylauristatin norephedrine or monomethylauristatin phenylalanine molecules.

Examples of antibodies that can be used in the compositions and methods of the invention, in some embodiments, are provided herein. In another embodiment, the antibody or antigen-binding fragment thereof is a monoclonal antibody or antigen-binding fragment thereof that specifically binds PSMA. In yet another embodiment, the antibody or antigen-binding fragment thereof is a monoclonal antibody or antigen-binding fragment thereof that specifically binds an extracellular domain of PSMA. In a further embodiment, the antibody or antigen-binding fragment thereof is a monoclonal antibody or antigen-binding fragment thereof that specifically binds to a conformational epitope of PSMA.

In some embodiments, the antibody or antigen-binding fragment thereof (i) competitively inhibits the specific binding of a second antibody to its target epitope on PSMA, or (ii) binds to an epitope on PSMA defined by an antibody selected from the group consisting of PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1 and Abgenix 4.152.1. In other embodiments, the antibody or antigen-binding fragment thereof binds to an epitope on PSMA defined by an antibody selected from the group consisting of antibodies comprising (a) a heavy chain encoded by a nucleic acid molecule comprising a coding region or regions of a nucleotide sequence selected from the group consisting of nucleotide sequences set forth as SEQ ID NOs: 2-7, and (b) a light chain encoded by a nucleic acid molecule comprising a coding region or regions of a nucleotide sequence selected from the group consisting of nucleotide sequences set forth as SEQ ID NOs: 8-13.

In some embodiments, the second antibody is selected from the group consisting of PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1, Abgenix 4.152.1 and antibodies comprising (a) a heavy chain encoded by a nucleic acid molecule comprising a coding region or regions of a nucleotide sequence selected from the group consisting of nucleotide sequences set forth as SEQ ID NOs: 2-7, and (b) a light chain encoded by a nucleic acid molecule comprising a coding region or regions of a nucleotide sequence selected from the group consisting of nucleotide sequences set forth as SEQ ID NOs: 8-13.

In other embodiments, the second antibody is selected from the group consisting of AB-PG1-XG1-006, AB-PG1-XG1-026 and antibodies comprising (a) a heavy chain encoded by a nucleic acid molecule comprising a coding region or regions of a nucleotide sequence selected from the group consisting of nucleotide sequences set forth as SEQ ID NOs: 2 and 3, and (b) a light chain encoded by a nucleic acid molecule comprising a coding region or regions of a nucleotide sequence selected from the group consisting of nucleotide sequences set forth as SEQ ID NOs: 8 and 9. In one embodiment, the second antibody comprises (a) a heavy chain encoded by a nucleic acid molecule comprising a coding region or regions of a nucleotide sequence set forth as SEQ ID NO: 2, and (b) a light chain encoded by a nucleic acid molecule comprising a coding region or regions of a nucleotide sequence set forth as SEQ ID NO: 8. In a further embodiment, the second antibody comprises (a) a heavy chain encoded by a nucleic acid molecule comprising a coding region or regions of a nucleotide sequence set forth as SEQ ID NO: 3, and (b) a light chain encoded by a nucleic acid molecule comprising a coding region or regions of a nucleotide sequence set forth as SEQ ID NO: 9.

In some embodiments, the antibody of the antibody-drug conjugate is an antibody encoded by a nucleic acid molecule comprising a nucleotide sequence that is at least 90% identical to a nucleotide sequence encoding an antibody selected from the group consisting of AB-PG1-XG1-006, AB-PG1-XG1-026 and antibodies comprising (a) a heavy chain encoded by a nucleic acid molecule comprising a coding region or regions of a nucleotide sequence selected from the group consisting of nucleotide sequences set forth as SEQ ID NOs: 2 and 3, and (b) a light chain encoded by a nucleic acid molecule comprising a coding region or regions of a nucleotide sequence selected from the group consisting of nucleotide sequences set forth as SEQ ID NOs: 8 and 9. In one embodiment, the antibody is encoded by a nucleic acid molecule comprising a nucleotide sequence that is at least 95% identical. In another embodiment, the antibody is encoded by a nucleic acid molecule comprising a nucleotide sequence that is at least 97% identical. In yet another embodiment, the antibody is encoded by a nucleic acid molecule comprising a nucleotide sequence that is at least 98% identical. In a further embodiment, the antibody is encoded by a nucleic acid molecule comprising a nucleotide sequence that is at least 99% identical.

In other embodiments, the antibody or antigen-binding fragment thereof of the antibody-drug conjugates provided herein is AB-PG1-XG1-006, AB-PG1-XG1-026 or an antigen-binding fragment thereof. In still other embodiments, the antibody or antigen-binding fragment thereof is selected from the group consisting of antibodies comprising (a) a heavy chain encoded by a nucleic acid molecule comprising a coding region or regions of a nucleotide sequence selected from the group consisting of nucleotide sequences set forth as SEQ ID NOs: 2 and 3, and (b) a light chain encoded by a nucleic acid molecule comprising a coding region or regions of a nucleotide sequence selected from the group consisting of nucleotide sequences set forth as SEQ ID NOs: 8 and 9, and antigen-binding fragments thereof. In one embodiment, the antibody or antigen-binding fragment thereof comprises (a) a heavy chain encoded by a nucleic acid molecule comprising a coding region or regions of a nucleotide sequence set forth as SEQ ID NO: 2, and (b) a light chain encoded by a nucleic acid molecule comprising a coding region or regions of a nucleotide sequence set forth as SEQ ID NO: 8, and antigen-binding fragments thereof. In another embodiment, the antibody or antigen-binding fragment thereof comprises (a) a heavy chain encoded by a nucleic acid molecule comprising a coding region or regions of a nucleotide sequence set forth as SEQ ID NO: 3, and (b) a light chain encoded by a nucleic acid molecule comprising a coding region or regions of a nucleotide sequence set forth as SEQ ID NO: 9, and antigen-binding fragments thereof.

In some embodiments, the antibody or antigen-binding fragment thereof is IgG1, IgG2, IgG3, IgG4, IgM, IgA1, IgA2, IgAsec, IgD, IgE or has immunoglobulin constant and/or variable domain of IgG1, IgG2, IgG3, IgG4, IgM, IgA1, IgA2, IgAsec, IgD or IgE.

In further embodiments, the antibody is a monoclonal antibody. In still other embodiments, the antibody is a humanized antibody. In yet other embodiments, the antibody is a human antibody. In still other embodiments, the antibody is a recombinant antibody. In further embodiments, the antibody is a chimeric antibody. In still further embodiments, the antibody is a bispecific or multispecific antibody. In yet other embodiments, the antibody is a single chain antibody.

In other embodiments, the antigen-binding fragment is a Fab fragment, a F(ab′)₂ fragment or a Fv fragment. In yet other embodiments, the antigen-binding fragment is a CDR3-containing fragment.

In some embodiments, the monomethylauristatin norephedrine (MMAE) or monomethylauristatin phenylalanine (MMAF) is conjugated to the antibody or antigen-binding fragment thereof with a compound of the formula (Formula 1) -Aₙ-Yₘ—Zₘ—Xₙ—Wₙ—, wherein A is a carboxylic acyl unit; Y is an amino acid; Z is an amino acid; X and W are each a self-immolative spacer; n is an integer of 0 or 1; and m is an integer of 0 or 1, 2, 3, 4, 5 or 6. In some embodiments, the conjugate of the present invention is represented by the formula (Formula 2): L-{Aₙ-Yₘ—Zₘ—Xₙ—Wₙ-D}ₚ wherein L is an antibody or antigen-binding fragment thereof that binds PSMA, D is MMAE or MMAF and p is an integer of 1, 2, 3, 4, 5, 6, 7 or 8. The rest of the components of the conjugate are as defined immediately above.

In one embodiment, the carboxylic unit “Aₙ” is linked to the antibody or antigen-binding fragment thereof via a sulfur atom derived from the antibody or antigen-binding fragment thereof:

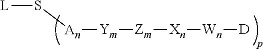

In one embodiment, A is

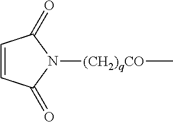

in which q is 1-10. Therefore, in one embodiment, the conjugate of Formula 2 is:

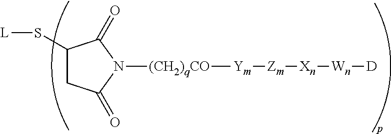

wherein L, Y, Z, X, W, D, n, m, q and p are as previously defined.

In another embodiment, A is 4-(N-succinimidomethyl)cyclohexane-1-carbonyl, m-succinimidobenzoyl, 4-(p-succinimidophenyl)-butyryl, 4-(2-acetamido)benzoyl, 3-thiopropionyl, 4-(1-thioethyl)-benzoyl, 6-(3-thiopropionylamido)-hexanoyl or maleimide caproyl. In a further embodiment, A is maleimide caproyl.

In another embodiment, Y is alanine, valine, leucine, isoleucine, methionine, phenylalanine, tryptophan or proline. In yet another embodiment, Y is valine. In a further embodiment, Z is lysine, lysine protected with acetyl or formyl, arginine, arginine protected with tosyl or nitro groups, histidine, ornithine, ornithine protected with acetyl or formyl, or citrulline. In still a further embodiment, Z is citrulline. In one embodiment Yₘ—Zₘ is valine-citrulline. In another embodiment, Yₘ—Zₘ is a protein sequence which is selectively cleavable by a protease.

In a further embodiment, X is a compound having the formula

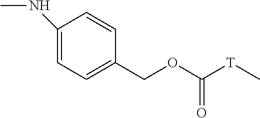

in which T is O, N, or S. In another embodiment, X is a compound having the formula —HN—R¹—COT in which R¹ is C₁-C₅ alkyl, T is O, N or S. In a further embodiment, X is a compound having the formula

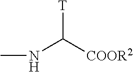

in which T is O, N, or S, R² is H or C₁-C₅ alkyl. In one embodiment, X is p-aminobenzylcarbamoyloxy. In another embodiment, X is p-aminobenzylalcohol. In a further embodiment, X is p-aminobenzylcarbamate. In yet a further embodiment, X is p-aminobenzyloxycarbonyl. In another embodiment, X is γ-aminobutyric acid; α,α-dimethyl γ-aminobutyric acid or β,β-dimethyl γ-aminobutyric acid.

In some embodiments, W is

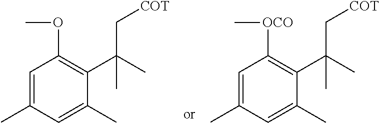

in which T is O, S or N.

In other embodiments, m and n are 0.

In one embodiment, the antibody-drug conjugate is AB-PG1-XG1-006-maleimide caproyl-valine-citrulline-p-aminobenzyloxycarbonyl-monomethylauristatin norephedrine. In another embodiment, the antibody-drug conjugate is AB-PG1-XG1-006-maleimide caproyl-valine-citrulline-p-aminobenzyloxycarbonyl-monomethylauristatin phenylalanine. In a further embodiment, the antibody-drug conjugate is AB-PG1-XG1-006-maleimide caproyl-monomethylauristatin phenylalanine. In another embodiment, the antibody-drug conjugate is AB-PG1-XG1-026-maleimide caproyl-valine-citrulline-p-aminobenzyloxycarbonyl-monomethylauristatin norephedrine. In yet another embodiment, the antibody-drug conjugate is AB-PG1-XG1-026-maleimide caproyl-valine-citrulline-p-aminobenzyloxycarbonyl-monomethylauristatin phenylalanine. In a further embodiment, the antibody-drug conjugate is AB-PG1-XG1-026-maleimide caproyl-monomethylauristatin phenylalanine. In another embodiment, the antibody-drug conjugate is a PSMA-binding antibody or antigen-binding fragment thereof conjugated to the compound as shown in FIG. 6A, FIG. 6B or FIG. 6C.

In some embodiments, the antibody-drug conjugate binds live cells. In one embodiment, the cell is a tumor cell. In another embodiment, the tumor cell is a prostate tumor cell. In a further embodiment, the tumor cell is a cell of the neovasculature of a non-prostate tumor. In other embodiments, the antibody-drug conjugate does not require cell lysis to bind PSMA. In still other embodiments, the antibody-drug conjugate leads to cell-cycle arrest. In yet further embodiments, the antibody-drug conjugate inhibits the growth of PSMA-expressing cells. In one embodiment, the antibody-drug conjugate mediates specific cell killing of PSMA-expressing cells with an IC₅₀ of less than 1×10⁻¹⁰M. In another embodiment, the IC₅₀ is less than 1×10⁻¹¹M. In yet another embodiment, the IC₅₀ is less than 1×10⁻¹²M. In a further embodiment, the antibody-drug conjugate mediates specific cell killing of PSMA-expressing cells with an IC₅₀ of 11 to 208×10⁻¹²M. In still a further embodiment, the antibody-drug conjugate mediates specific cell killing of PSMA-expressing cells with an IC₅₀ of 42 to 208×10⁻¹²M. In yet a further embodiment, the antibody-drug conjugate mediates specific cell killing of PSMA-expressing cells with an IC₅₀ of 60 to 208×10⁻¹²M. In another embodiment, the antibody-drug conjugate mediates specific cell killing of PSMA-expressing cells with an IC₅₀ of 65 to 208×10⁻¹²M. In one embodiment, the antibody-drug conjugate mediates specific cell killing of PSMA-expressing cells with an IC₅₀ of 11×10⁻¹²M. In another embodiment, the antibody-drug conjugate mediates specific cell killing of PSMA-expressing cells with an IC₅₀ of 42×10⁻¹²M. In still another embodiment, the antibody-drug conjugate mediates specific cell killing of PSMA-expressing cells with an IC₅₀ of 60×10⁻¹²M. In a further embodiment, the antibody-drug conjugate mediates specific cell killing of PSMA-expressing cells with an IC₅₀ of 83×10⁻¹²M.

In another embodiment, the antibody-drug conjugate, when administered to mice with a regimen of q4d×6 at a dose of 6 mg/kg effects a cure rate of at least 20%, 30%, 40% or 50%. In one embodiment, the cure rate is 20%, 30%, 40%, 50%, 60%, 70%, 80% or more. In one embodiment, the mice are those that are a model of androgen-independent human prostate cancer. In another embodiment, the mice are nude mice engrafted with C4-2 cells intramuscularly in the left hind-leg. In a further embodiment, the mice are those as provided in the Examples.

In some embodiments, the antibody-drug conjugate is bound to a label. In other embodiments, the label is a fluorescent label, an enzyme label, a radioactive label, a nuclear magnetic resonance active label, a luminescent label or a chromophore label.

In some embodiments, the antibody-drug conjugate is packaged in lyophilized form. In other embodiments, the antibody-drug conjugate is packaged in an aqueous medium. In further embodiments, the antibody-drug conjugate is in a sterile form.

Also provided herein are compositions comprising one or more antibody-drug conjugates. In some embodiments, the composition comprises two or more different antibody-drug conjugates. In other embodiments, a composition comprising one or more antibody-drug conjugates and one or more unconjugated anti-PSMA antibodies is provided.

In some embodiments, the composition further comprises a pharmaceutically acceptable carrier, excipient or stabilizer. In other embodiments, the composition further comprises an antitumor agent, an immunostimulatory agent, an immunomodulator, a corticosteroid or a combination thereof. In one embodiment, the antitumor agent is a cytotoxic agent, an agent that acts on tumor neovasculature or a combination thereof. In another embodiment, the antitumor agent is docetaxel. In still another embodiment, the immunomodulator is a cytokine, chemokine, adjuvant or a combination thereof. In yet another embodiment, the immunostimulatory agent is interleukin-2, α-interferon, γ-interferon, tumor necrosis factor-α, immunostimulatory oligonucleotides or a combination thereof. In a further embodiment, the corticosteroid is prednisone or hydrocortisone. In still a further embodiment, the composition comprises prednisone and docetaxel.

A variety of methods for using the antibody-drug conjugates and compositions of the invention are provided. In one embodiment, a method for inhibiting the growth of a PSMA-expressing cell comprising contacting the PSMA-expressing cell with an amount of an antibody-drug conjugate effective to inhibit the growth of the PSMA-expressing cell is provided. In another embodiment, a method for effecting cell-cycle arrest in a PSMA-expressing cell comprising contacting the PSMA-expressing cell with an amount of an antibody-drug conjugate effective to lead to cell-cycle arrest in the PSMA-expressing cell is provided. In still another embodiment, a method for treating a PSMA-mediated disease comprising administering to a subject having a PSMA-mediated disease an amount of an antibody-drug conjugate effective to treat the PSMA-mediated disease is provided. In a further embodiment, a method for inhibiting the growth of a tumor comprising contacting PSMA-expressing cells of the neovasculature of the tumor with an amount of an antibody-drug conjugate effective to inhibit the growth of the tumor is provided.

In one embodiment, the PSMA-mediated disease is cancer. In another embodiment, the cancer is a prostate cancer. In yet another embodiment, the cancer is a non-prostate cancer. In some embodiments, the non-prostate cancer is bladder cancer, pancreatic cancer, lung cancer, kidney cancer, sarcoma, breast cancer, brain cancer, neuroendocrine carcinoma, colon cancer, testicular cancer or melanoma.

In some embodiments, the method further comprises co-administering another therapeutic agent to treat the PSMA-mediated disease. In other embodiments, the method further comprises contacting PSMA-expressing cells with another therapeutic agent. In some embodiments, the other therapeutic agent is administered before, during or after the administration of the antibody-drug conjugate. In one embodiment, the other therapeutic agent is an antitumor agent, an immunostimulatory agent, an immunomodulator, a corticosteroid or a combination thereof. In another embodiment, the antitumor agent is a cytotoxic agent, an agent that acts on tumor neovasculature or a combination thereof. In yet another embodiment, the antitumor agent is docetaxel. In still another embodiment, the immunomodulator is a cytokine, chemokine, adjuvant or a combination thereof. In yet another embodiment, the immunostimulatory agent is interleukin-2, α-interferon, γ-interferon, tumor necrosis factor-α, immunostimulatory oligonucleotides or a combination thereof. In a further embodiment, the corticosteroid is prednisone or hydrocortisone. In one embodiment, the therapeutic agent is a vaccine. In another embodiment, the vaccine immunizes the subject against PSMA. In another embodiment, the method further comprises administering still another therapeutic agent. In one embodiment, the still another therapeutic agent is prednisone. In one embodiment, therefore, both docetaxel and prednisone are administered.

The PSMA-expressing cell is, in some embodiments, a prostate tumor cell or a cell of the neovasculature of a non-prostate tumor. In some embodiments, the PSMA-expressing cell is an androgen-dependent cell or an androgen-independent cell.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE FIGURES

FIGS. 1A-1B are graphs that show the percent internalization and total binding of ¹¹¹In-labeled PSMA mAb on C4-2 cells. C4-2 cells were incubated with ¹¹¹In-labeled mAb at 37° C., 5% CO₂. At the designated times, cells were washed to remove unbound mAb, and surface bound mAb was stripped using low pH buffer. The radioactivity (counts per minute (CPM)) of the low pH eluate and cell pellet was counted separately using a gamma counter. Percent internalization (FIG. 1A) was calculated as the CPM cell pellet/(CPM cell pellet+CPM low pH eluate)×100. Total binding (FIG. 1B) represents the CPM of the cell pellet plus the CPM of the low pH eluate.

FIG. 2 is a graph showing the binding of PSMA mAb and ADC to 3T3™-PSMA cells. 3T3™-PSMA cells were incubated with increasing concentrations of the PSMA mAb (filled squares), PSMA ADC (open squares) or isotype-control ADC (open triangles). Cells were incubated on ice for 1 h and washed to remove unbound mAb or ADC. The cells were then incubated with goat anti-human IgG-FITC, washed again and examined by flow cytometry. The mean fluorescence intensities (MFIs) are plotted as a function of mAb or ADC concentration.

FIGS. 3A-3B are graphs showing the in vitro cytotoxicity of the PSMA ADC and control ADC on PSMA-positive and PSMA-negative prostate cancer cell lines. PSMA-positive C4-2 cells (FIG. 3A) and PSMA-negative PC-3™ cells (FIG. 3B) in 96-well microplates were exposed to ADCs at various concentrations. After 96 hours, cell survival in treated and untreated cultures was assayed using Alamar Blue.

FIGS. 4A-4B are graphs showing the Kaplan-Meier survival and serum PSA levels in a xenograft study. Nude mice were implanted intramuscularly with C4-2 cells, randomly assigned to treatment groups (6 mice per group) according to serum PSA on day 17 and then treated q4d×3 with PSMA ADC or vehicle. FIG. 4A shows the survival of animals treated with 0 (vehicle control, dashed line), 2 mg/kg (thin solid line) and 10 mg/kg PSMA ADC. FIG. 4B provides the mean PSA values over 30 days in mice treated with 0 (filled columns), 2 mg/kg (striped columns) and 10 mg/kg (open columns) PSMA ADC. The day 30 data for the control group include day 27 evaluations for two mice which did not survive 30 days.

FIG. 5 shows Kaplan-Meier survival curves of animals treated in another xenograft study. Nude mice were implanted intramuscularly with C4-2 cells, randomly assigned to treatment groups (5 mice per group) according to serum PSA on day 14 and then treated q4d×6 with PSMA ADC and controls. Mice were treated with 0 (vehicle control, filled circles), 6 mg/kg unmodified PSMA mAb (filled triangles), 6 mg/kg control ADC (open triangles), 3 mg/kg PSMA ADC (open squares) and 6 mg/kg PSMA ADC (filled squares).

FIGS. 6A-6C show the chemical structures of three different drug-linkers. FIG. 6A provides the structure of vcMMAE (maleimidocaproyl-valine-citrulline-p-aminobenzyloxycarbonyl-monomethylauristatin E). FIG. 6B provides the structure of vcMMAF (maleimidocaproyl-valine-citrulline-p-aminobenzyloxycarbonyl-monomethylauristatin F). FIG. 6C provides the structure of mcMMAF (maleimidocaproyl-monomethylauristatin F).

FIGS. 7A-7C demonstrate the in vitro cytotoxicity of the PSMA ADCs (vcMMAE (FIG. 7A), vcMMAF (FIG. 7B), mcMMAF (FIG. 7C)) on PSMA-positive (C4-2) and PSMA-negative (PC-3™) prostate cancer cell lines. The cells in 96-well microplates were exposed to ADCs at various concentrations. After 4 days, cell survival in treated and untreated cultures was assayed using Alamar Blue.

FIG. 8 illustrates effects of PSMA ADC on cell cycle. In each panel, the left peak corresponds to G₁ phase and the right peak to G₂/M phase. The percent of cells in G₂/M increased markedly upon treatment with the PSMA ADC, consistent with an arrest in cell division that occurs after DNA synthesis. The PSMA ADC did not affect cycling of parental 3T3™ cells.

FIG. 9 shows the results from a comparison of PSMA ADCs vcMMAE v. vcMMAF.

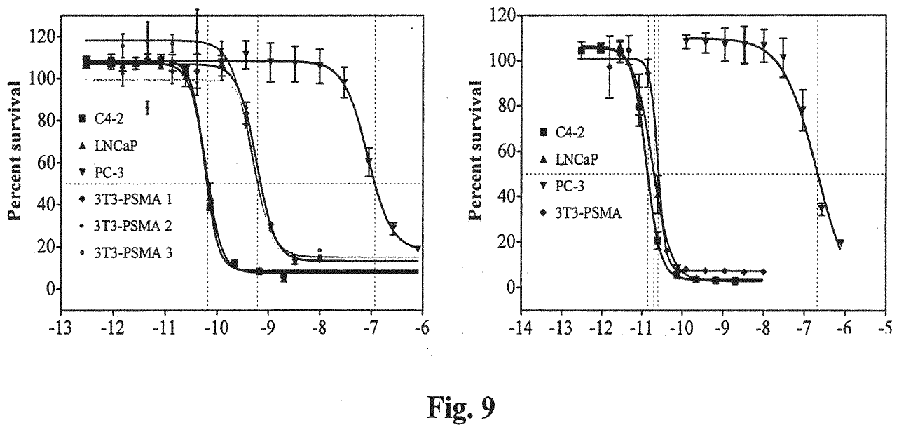

### DETAILED DESCRIPTION OF THE INVENTION

The present invention relates, in part, to the surprising discovery that ADCs comprising a PSMA-binding antibody or antigen-binding fragment thereof conjugated to MMAE (also referred to herein as monomethylauristatin E and monomethylauristatin norephedrine) or MMAF (also referred to herein as monomethylauristatin F and monomethylauristatin phenylalanine) are particularly useful for killing PSMA-expressing cells. The ADCs can have a PC-3™ cell to C4-2 or LNCaP™ cell selectivity of at least 250. In some embodiments, the ADCs exhibit certain levels of cell killing (of PSMA-expressing cells), e.g., IC₅₀ values that are at or near picomolar concentrations. In other embodiments, the ADCs effect a cure rate of at least 20%, 30%, 40% or 50% in mice treated with the ADC with a regimen of q4d×6 at a dose of 6 mg/kg. Compositions of and methods of using these ADCs are, therefore, provided. In some embodiments, the mice are those as provided in the Examples. In one embodiment, the mice are those that are a model of androgen-independent human prostate cancer. In another embodiment, the mice are nude mice engrafted with C4-2 cells intramuscularly in the left hind-leg.

The antibodies or antigen-binding fragments thereof of the ADCs are any antibody or antigen-binding fragment thereof that binds PSMA. In one embodiment the antibody or an antigen-binding fragment thereof specifically binds PSMA (e.g., specifically binds an extracellular domain of PSMA, specifically binds a conformational epitope of PSMA, etc.) and can competitively inhibit the specific binding of a second antibody to its target epitope on PSMA, wherein the second antibody is selected from the group consisting of PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1, Abgenix 4.152.1 and antibodies comprising (a) a heavy chain encoded by a nucleic acid molecule comprising a coding region or regions of a nucleotide sequence selected from the group consisting of nucleotide sequences set forth as SEQ ID NOs: 2-7, and (b) a light chain encoded by a nucleic acid molecule comprising a coding region or regions of a nucleotide sequence selected from the group consisting of nucleotide sequences set forth as SEQ ID NOs: 8-13. The second antibody, therefore, include any of the antibodies produced by the hybridomas or encoded by the plasmids shown below in Table 1. These hybridomas and plasmids were deposited pursuant to, and in satisfaction of, the requirements of the Budapest Treaty on the International Recognition of the Deposit of Microorganisms for the Purposes of Patent Procedure with the American Type Culture Collection (“ATCC”) as an International Depository Authority and given the Patent Deposit Designations shown above and in Table 1.

TABLE 1

| | | Patent | |
|---|---|---|---|
| | | Deposit | Date of |
| Antibody | Hybridoma/Plasmid | Designation | Deposit |
| PSMA 3.7 | PSMA 3.7 | PTA-3257 | Apr. 5, 2001 |
| PSMA 3.9 | PSMA 3.9 | PTA-3258 | Apr. 5, 2001 |
| PSMA 3.11 | PSMA 3.11 | PTA-3269 | Apr. 10, 2001 |
| PSMA 5.4 | PSMA 5.4 | PTA-3268 | Apr. 10, 2001 |
| PSMA 7.1 | PSMA 7.1 | PTA-3292 | Apr. 18, 2001 |
| PSMA 7.3 | PSMA 7.3 | PTA-3293 | Apr. 18, 2001 |
| PSMA 10.3 | PSMA 10.3 | PTA-3347 | May 1, 2001 |
| | PSMA 10.3 HC in | PTA-4413 | May 29, 2002 |
| | pcDNA (SEQ ID NO: 7) | | |
| | PSMA 10.3 Kappa in | PTA-4414 | May 29, 2002 |
| | pcDNA (SEQ ID NO: 13) | | |
| PSMA 1.8.3 | PSMA 1.8.3 | PTA-3906 | Dec. 5, 2001 |
| PSMA A3.1.3 | PSMA A3.1.3 | PTA-3904 | Dec. 5, 2001 |
| PSMA A3.3.1 | PSMA A3.3.1 | PTA-3905 | Dec. 5, 2001 |
| Abgenix 4.248.2 | Abgenix 4.248.2 | PTA-4427 | Jun. 4, 2002 |
| Abgenix 4.360.3 | Abgenix 4.360.3 | PTA-4428 | Jun. 4, 2002 |
| Abgenix 4.7.1 | Abgenix 4.7.1 | PTA-4429 | Jun. 4, 2002 |
| Abgenix 4.4.1 | Abgenix 4.4.1 | PTA-4556 | Jul. 18, 2002 |
| Abgenix 4.177.3 | Abgenix 4.177.3 | PTA-4557 | Jul. 18, 2002 |
| Abgenix 4.16.1 | Abgenix 4.16.1 | PTA-4357 | May 16, 2002 |
| Abgenix 4.22.3 | Abgenix 4.22.3 | PTA-4358 | May 16, 2002 |
| Abgenix 4.28.3 | Abgenix 4.28.3 | PTA-4359 | May 16, 2002 |
| Abgenix 4.40.2 | Abgenix 4.40.2 | PTA-4360 | May 16, 2002 |
| Abgenix 4.48.3 | Abgenix 4.48.3 | PTA-4361 | May 16, 2002 |
| Abgenix 4.49.1 | Abgenix 4.49.1 | PTA-4362 | May 16, 2002 |
| Abgenix 4.209.3 | Abgenix 4.209.3 | PTA-4365 | May 16, 2002 |
| Abgenix 4.219.3 | Abgenix 4.219.3 | PTA-4366 | May 16, 2002 |
| Abgenix 4.288.1 | Abgenix 4.288.1 | PTA-4367 | May 16, 2002 |
| Abgenix 4.333.1 | Abgenix 4.333.1 | PTA-4368 | May 16, 2002 |
| Abgenix 4.54.1 | Abgenix 4.54.1 | PTA-4363 | May 16, 2002 |
| Abgenix 4.153.1 | Abgenix 4.153.1 | PTA-4388 | May 23, 2002 |
| Abgenix 4.232.3 | Abgenix 4.232.3 | PTA-4389 | May 23, 2002 |
| Abgenix 4.292.3 | Abgenix 4.292.3 | PTA-4390 | May 23, 2002 |
| Abgenix 4.304.1 | Abgenix 4.304.1 | PTA-4391 | May 23, 2002 |
| AB-PG1-XG1-006 | AB-PG1-XG1-006 Heavy | PTA-4403 | May 29, 2002 |
| | Chain (SEQ ID NO: 2) | | |
| | AB-PG1-XG1-006 Light | PTA-4404 | |
| | Chain (SEQ ID NO: 8) | | |
| AB-PG1-XG1-026 | AB-PG1-XG1-026 Heavy | PTA-4405 | May 29, 2002 |
| | Chain (SEQ ID NO: 3) | | |
| | AB-PG1-XG1-026 Light | PTA-4406 | |
| | Chain (SEQ ID NO: 9) | | |
| AB-PG1-XG1-051 | AB-PG1-XG1-051 Heavy | PTA-4407 | May 29, 2002 |
| | Chain (SEQ ID NO: 4) | | |
| | AB-PG1-XG1-051 Light | PTA-4408 | |
| | Chain (SEQ ID NO: 10) | | |
| AB-PG1-XG1-069 | AB-PG1-XG1-069 Heavy | PTA-4409 | May 29, 2002 |
| | Chain (SEQ ID NO: 5) | | |
| | AB-PG1-XG1-069 Light | PTA-4410 | |
| | Chain (SEQ ID NO: 11) | | |
| AB-PG1-XG1-077 | AB-PG1-XG1-077 Heavy | PTA-4411 | May 29, 2002 |
| | Chain (SEQ ID NO: 6) | | |
| | AB-PG1-XG1-077 Light | PTA-4412 | |
| | Chain (SEQ ID NO: 12) | | |

To determine competitive inhibition, a variety of assays known to one of ordinary skill in the art can be employed. For example, cross-competition assays can be used to determine if an antibody or antigen-binding fragment thereof competitively inhibits binding to PSMA by another antibody or antigen-binding fragment thereof. These include cell-based methods employing flow cytometry or solid phase binding analysis. Other assays that evaluate the ability of antibodies or antigen-binding fragments thereof to cross-compete for PSMA molecules that are not expressed on the surface of cells, in solid phase or in solution phase, also can be used.

In some embodiments, the antibodies or antigen-binding fragments thereof competitively inhibit the specific binding of a second antibody to its target epitope on PSMA by at least about 10%, 20%, 30%, 40%, 50%, 60%, 70%, 80%, 90%, 95%, or 99%. Inhibition can be assessed at various molar ratios or mass ratios; for example competitive binding experiments can be conducted with a 2-fold, 3-fold, 4-fold, 5-fold, 7-fold, 10-fold or more molar excess of a first antibody or antigen-binding fragment thereof over a second antibody or antigen-binding fragment thereof.

In another embodiment the antibody or an antigen-binding fragment thereof specifically binds to an epitope on PSMA defined by an antibody selected from the group consisting of PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, 4.248.2, 4.360.3, 4.7.1, 4.4.1, 4.177.3, 4.16.1, 4.22.3, 4.28.3, 4.40.2, 4.48.3, 4.49.1, 4.209.3, 4.219.3, 4.288.1, 4.333.1, 4.54.1, 4.153.1, 4.232.3, 4.292.3, 4.304.1, 4.78.1, and 4.152.1. PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1, Abgenix 4.152.1 and antibodies comprising (a) a heavy chain encoded by a nucleic acid molecule comprising a coding region or regions of a nucleotide sequence selected from the group consisting of nucleotide sequences set forth as SEQ ID NOs: 2-7, and (b) a light chain encoded by a nucleic acid molecule comprising a coding region or regions of a nucleotide sequence selected from the group consisting of nucleotide sequences set forth as SEQ ID NOs: 8-13. The antibodies or antigen-binding fragments of the ADCs, therefore, include those that specifically bind to an epitope on PSMA defined by the antibodies produced by the hybridomas or encoded by the plasmids provided above in Table 1.

To determine the epitope, one can use standard epitope mapping methods known in the art. For example, fragments (peptides) of PSMA antigen (e.g., synthetic peptides) that bind the antibody can be used to determine whether a candidate antibody or antigen-binding fragment thereof binds the same epitope. For linear epitopes, overlapping peptides of a defined length (e.g., 8 or more amino acids) are synthesized. The peptides can be offset by 1 amino acid, such that a series of peptides covering every 8 amino acid fragment of the PSMA protein sequence are prepared. Fewer peptides can be prepared by using larger offsets, e.g., 2 or 3 amino acids. In addition, longer peptides (e.g., 9-, 10- or 11-mers) can be synthesized. Binding of peptides to antibodies or antigen-binding fragments can be determined using standard methodologies including surface plasmon resonance (BIACORE) and ELISA assays. For examination of conformational epitopes, larger PSMA fragments can be used. Other methods that use mass spectrometry to define conformational epitopes have been described and can be used (see, e.g., Baerga-Ortiz et al., Protein Science 11:1300-1308, 2002 and references cited therein). Still other methods for epitope determination are provided in standard laboratory reference works, such as Unit 6.8 (“Phage Display Selection and Analysis of B-cell Epitopes”) and Unit 9.8 (“Identification of Antigenic Determinants Using Synthetic Peptide Combinatorial Libraries”) of Current Protocols in Immunology, Coligan et al., eds., John Wiley & Sons. Epitopes can be confirmed by introducing point mutations or deletions into a known epitope, and then testing binding with one or more antibodies or antigen-binding fragments to determine which mutations reduce binding of the antibodies or antigen-binding fragments.

In particular embodiments, the antibodies of the ADCs, or from which the antigen-binding fragments of the ADCs are derived, are those produced by hybridomas referred to herein as PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1, and Abgenix 4.152.1, respectively. In other embodiments, the antibodies are those encoded by the plasmids shown in Table 1. In still other particular embodiments, the antibodies are those that comprise a heavy chain encoded by a nucleic acid molecule comprising the heavy chain coding region or regions of a nucleotide sequence selected from the group consisting of nucleotide sequences set forth as SEQ ID NOs: 2-7, and a light chain encoded by a nucleic acid molecule comprising the light chain coding region or regions of a nucleotide sequence selected from the group consisting of nucleotide sequences set forth as SEQ ID NOs: 8-13.

As used herein, the names of the deposited hybridomas or plasmids may be used interchangeably with the names of the antibodies. It would be clear to one of ordinary skill in the art when the name is intended to refer to the antibody or when it refers to the plasmids or hybridomas that encode or produce the antibodies, respectively. Additionally, the antibody names may be an abbreviated form of the name shown in Table 1. For instance, antibody AB-PG1-XG1-006 may be referred to as AB-PG1-XG1-006, PG1-XG1-006, XG1-006, 006, etc. In another example, the antibody name PSMA 4.232.3 may be referred to as PSMA 4.232.1, 4.232.3, 4.232.1, 4.232, etc. It is intended that all of the variations in the name of the antibody refer to the same antibody and not a different one.

The antibodies of the ADCs, or from which the antigen-binding fragments of the ADCs are derived, include those encoded by particular sets of heavy and light chain sequences. In one embodiment, the antibody (AB-PG1-XG1-006) is encoded by a nucleic acid molecule which comprises a coding region or regions of the nucleic acid sequences set forth as SEQ ID NOs: 2 and 8. In another embodiment, the antibody (AB-PG1-XG1-026) is encoded by a nucleic acid molecule which comprises a coding region or regions of the nucleic acid sequences set forth as SEQ ID NOs: 3 and 9. In still another embodiment, the antibody (AB-PG1-XG1-051) is encoded by a nucleic acid molecule which comprises a coding region or regions of the nucleic acid sequences set forth as SEQ ID NOs: 4 and 10. In yet another embodiment, the antibody (AB-PG1-XG1-069) is encoded by a nucleic acid molecule which comprises a coding region or regions of the nucleic acid sequences set forth as SEQ ID NOs: 5 and 11. In another embodiment, the antibody (AB-PG1-XG1-077) is encoded by a nucleic acid molecule which comprises a coding region or regions of the nucleic acid sequences set forth as SEQ ID NOs: 6 and 12. In yet another embodiment, the antibody (PSMA 10.3) is encoded by a nucleic acid molecule which comprises a coding region or regions of the nucleic acid sequences set forth as SEQ ID NOs: 7 and 13. In other embodiments, the antibodies of the ADCs, or from which the antigen-binding fragments of the ADCs are derived, include a heavy chain variable region encoded by a nucleic acid molecule comprising a coding region or regions of a nucleotide sequence selected from the group consisting of nucleotide sequences set forth as SEQ ID NOs: 14, 18, 22, 26 and 30, and a light chain variable region encoded by a nucleic acid molecule comprising a coding region or regions of a nucleotide sequence selected from the group consisting of nucleotide sequences set forth as SEQ ID NOs: 16, 20, 24, 28 and 32. In one embodiment, the antibody (AB-PG1-XG1-006) includes an immunoglobulin variable sequence encoded by nucleic acid molecules which comprise a coding region or regions of the nucleic acid sequences set forth as SEQ ID NOs: 14 and 16. Likewise, the antibody can be one that includes an immunoglobulin variable sequence which comprises the amino acid sequences set forth as SEQ ID NOs: 15 and 17. In another embodiment, the antibody (AB-PG1-XG1-026) includes an immunoglobulin variable sequence encoded by nucleic acid molecules comprising a coding region or regions of nucleotide sequences set forth as SEQ ID NOs: 18 and 20 or includes an immunoglobulin variable sequence which comprises the amino acid sequences set forth as SEQ ID NOs 19 and 21. In still another embodiment, the antibody (AB-PG1-XG1-051) includes an immunoglobulin variable sequence encoded by the nucleic acid molecules comprising a coding region or regions of nucleotide sequences set forth as SEQ ID NOs: 22 and 24 or includes an immunoglobulin variable sequence which comprises the amino acid sequences set forth as SEQ ID NOs: 23 and 25. In yet another embodiment, the antibody (AB-PG1-XG1-069) includes an immunoglobulin variable sequence encoded by the nucleic acid molecules comprising a coding region or regions of nucleotide sequences set forth as SEQ ID NOs: 26 and 28 or includes an immunoglobulin variable sequence which comprises the amino acid sequences set forth as SEQ ID NOs: 27 and 29. In another embodiment, the antibody (AB-PG1-XG1-077) includes an immunoglobulin variable sequence encoded by the nucleic acid molecules comprising a coding region or regions of nucleotide sequences set forth as SEQ ID NOs: 30 and 32 or includes an immunoglobulin variable sequence which comprises the amino acid sequences set forth as SEQ ID NOs: 31 and 33. In other embodiments, the antibody includes a heavy chain variable region comprising an amino acid sequence selected from the group consisting of amino acid sequences set forth as: SEQ ID NOs: 15, 19, 23, 27 and 31, and a light chain variable region comprising an amino acid sequence selected from the group consisting of amino acid sequences set forth as: SEQ ID NOs: 17, 21, 25, 29 and 33.

As used herein, a “coding region” refers to a region of a nucleotide sequence that encodes a polypeptide sequence. Its use herein is consistent with the recognized meaning known in the art.

In certain embodiments, the antibodies of the ADCs, or from which the antigen-binding fragments of the ADCs are derived, are those that are encoded by nucleic acid molecules that are highly homologous to the foregoing nucleic acids. The homologous nucleic acid molecule can, in some embodiments, comprise a nucleotide sequence that is at least about 90% identical to the nucleotide sequence provided herein. In other embodiments, the nucleotide sequence is at least about 95% identical, at least about 97% identical, at least about 98% identical, or at least about 99% identical to a nucleotide sequence provided herein. The homology can be calculated using various, publicly available software tools well known to one of ordinary skill in the art. Exemplary tools include the BLAST system available from the website of the National Center for Biotechnology Information (NCBI) at the National Institutes of Health.

One method of identifying highly homologous nucleotide sequences is via nucleic acid hybridization. Thus, the invention also includes antibodies having the PSMA-binding properties and other functional properties described herein, which are encoded by nucleic acid molecules that hybridize under high stringency conditions to the foregoing nucleic acid molecules. Identification of related sequences can also be achieved using polymerase chain reaction (PCR) and other amplification techniques suitable for cloning related nucleic acid sequences. PCR primers can be selected to amplify portions of a nucleic acid sequence of interest, such as a CDR.

The term “high stringency conditions”, as used herein, refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. One example of high-stringency conditions is hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄ (pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, a membrane upon which the nucleic acid is transferred is washed, for example, in 2×SSC at room temperature and then at 0.1-0.5×SSC/0.1×SDS at temperatures up to 68° C.

As used herein, the term “antibody” refers to a glycoprotein comprising at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody as used herein, refers to one or more portions of an antibody that retain the ability to specifically bind to an antigen (i.e., PSMA). It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546) which consists of a V_{H} domain; and (vi) an isolated complementarity determining region (CDR). The CDRs, and in particular the CDR3 regions, and more particularly the heavy chain CDR3 contribute to antibody specificity. Because these CDR regions and in particular the CDR3 region confer antigen specificity on the antibody these regions may be incorporated into other antibodies or antigen-binding fragments to confer the identical antigen specificity onto that antibody or peptide. Furthermore, although the two domains of the Fv fragment, V and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

The antibodies, or antigen-binding fragments thereof, of the ADCs are, in some embodiments, isolated. “Isolated”, as used herein, is intended to refer to an antibody (or antigen-binding fragment thereof), which is substantially free of other antibodies (or antigen-binding fragments) having different antigenic specificities (e.g., an isolated antibody that specifically binds to PSMA is substantially free of antibodies that specifically bind antigens other than PSMA). An isolated antibody that specifically binds to an epitope, isoform or variant of PSMA may, however, have cross-reactivity to other related antigens, e.g., from other species (e.g., PSMA species homologs). Moreover, an isolated antibody (or antigen-binding fragment thereof) may be substantially free of other cellular material and/or chemicals. As used herein, “specific binding” refers to antibody binding to a predetermined antigen, in this case PSMA. Typically, the antibody binds with an affinity that is at least two-fold greater than its affinity for binding to a non-specific antigen (e.g., BSA, casein), which is an antigen other than PSMA, an isoform or variant of PSMA, or a closely-related antigen.

The antibodies encompass various antibody isotypes, such as IgG1, IgG2, IgG3, IgG4, IgM, IgA1, IgA2, IgAsec, IgD, IgE. As used herein, “isotype” refers to the antibody class (e.g., IgM or IgG1) that is encoded by heavy chain constant region genes. The antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)₂ fragment and a Fv fragment.

The antibodies of the ADCs, or from which the antigen-binding fragments of the ADCs are derived, are, in some embodiments monoclonal. The antibodies can be produced by a variety of techniques well known in the art. Monoclonal antibody production may be effected by techniques which are well known in the art. The term “monoclonal antibody”, as used herein, refers to a preparation of antibody molecules of single molecular composition. A monoclonal antibody displays a single binding specificity and affinity for a particular epitope. The process of monoclonal antibody production involves obtaining immune somatic cells with the potential for producing antibody, in particular B lymphocytes, which have been previously immunized with the antigen of interest either in vivo or in vitro and that are suitable for fusion with a B-cell myeloma line.

Mammalian lymphocytes typically are immunized by in vivo immunization of the animal (e.g., a mouse) with the desired protein or polypeptide. Such immunizations are repeated as necessary at intervals of up to several weeks to obtain a sufficient titer of antibodies. Once immunized, animals can be used as a source of antibody-producing lymphocytes. Following the last antigen boost, the animals are sacrificed and spleen cells removed. Mouse lymphocytes give a higher percentage of stable fusions with the mouse myeloma lines described herein. For example, of the BALB/c mouse. However, other mouse strains, rabbit, hamster, sheep and frog may also be used as hosts for preparing antibody-producing cells. See; Goding (in Monoclonal Antibodies: Principles and Practice, 2d ed., pp. 60-61, Orlando, Fla., Academic Press, 1986). In particular, mouse strains that have human immunoglobulin genes inserted in the genome (and which cannot produce mouse immunoglobulins) can be used. Examples include the HuMAb mouse strains produced by Medarex/GenPharm International, and the XenoMouse strains produced by Abgenix. Such mice produce fully human immunoglobulin molecules in response to immunization. In some embodiments, therefore, the ADCs comprise a fully human monoclonal antibody or an antigen-binding fragment thereof that binds PSMA.

Those antibody-producing cells that are in the dividing plasmablast stage fuse preferentially. Somatic cells may be obtained from the lymph nodes, spleens and peripheral blood of antigen-primed animals, and the lymphatic cells of choice depend to a large extent on their empirical usefulness in the particular fusion system. The antibody-secreting lymphocytes are then fused with (mouse) B cell myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Alternatively, human somatic cells capable of producing antibody, specifically B lymphocytes, are suitable for fusion with myeloma cell lines. While B lymphocytes from biopsied spleens, tonsils or lymph nodes of an individual may be used, the more easily accessible peripheral blood B lymphocytes can also be used. The lymphocytes may be derived from patients with diagnosed prostate carcinomas or another PSMA-expressing cancer. In addition, human B cells may be directly immortalized by the Epstein-Barr virus (Cole et al., 1995, Monoclonal Antibodies and Cancer Therapy, Alan R. Liss, Inc., pp. 77-96). Although somatic cell hybridization procedures can be used, in principle, other techniques for producing monoclonal antibodies can be employed such as viral or oncogenic transformation of B lymphocytes.

Myeloma cell lines suited for use in hybridoma-producing fusion procedures can be non-antibody-producing, have high fusion efficiency, and enzyme deficiencies that render them incapable of growing in certain selective media which support the growth of the desired hybridomas. Examples of such myeloma cell lines that may be used for the production of fused cell lines include P3-X63/Ag8, X63-Ag8.653, NS1/1.Ag 4.1, Sp2/0-Ag14, FO, NSO/U, MPC-11, MPC11-X45-GTG 1.7, S194/5XX0 Bul, all derived from mice; R210.RCY3, Y3-Ag 1.2.3, IR983F and 4B210 derived from rats and U-266, GM1500-GRG2, LICR-LON-HMy2, UC729-6, all derived from humans (Goding, in Monoclonal Antibodies: Principles and Practice, 2d ed., pp. 65-66, Orlando, Fla., Academic Press, 1986; Campbell, in Monoclonal Antibody Technology, Laboratory Techniques in Biochemistry and Molecular Biology Vol. 13, Burden and Von Knippenberg, eds. pp. 75-83, Amsterdam, Elseview, 1984).

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, Eur. J. Immunol. 6:511 (1976), which is hereby incorporated by reference).

In other embodiments, the antibodies of the ADCs, or from which the antigen-binding fragments of the ADCs are derived, are recombinant antibodies. The term “recombinant antibody”, as used herein, is intended to include antibodies that are prepared, expressed, created or isolated by recombinant means, such as antibodies isolated from an animal (e.g., a mouse) that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

In yet other embodiments, the antibodies are chimeric or humanized antibodies. As used herein, the term “chimeric antibody” refers to an antibody, that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for applications according to the invention.

According to an alternative embodiment, the monoclonal antibodies of the present invention can be modified to be in the form of a bispecific antibody, or a multispecific antibody. The term “bispecific antibody” is intended to include any agent, e.g., a protein, peptide, or protein or peptide complex, which has two different binding specificities which bind to, or interact with (a) a cell surface antigen and (b) an Fc receptor on the surface of an effector cell. The term “multispecific antibody” is intended to include any agent, e.g., a protein, peptide, or protein or peptide complex, which has more than two different binding specificities which bind to, or interact with (a) a cell surface antigen, (b) an Fc receptor on the surface of an effector cell, and (c) at least one other component. Accordingly, the antibodies include, but are not limited to, bispecific, trispecific, tetraspecific, and other multispecific antibodies which are directed to PSMA and to Fc receptors on effector cells. The term “bispecific antibodies” further includes diabodies. Diabodies are bivalent, bispecific antibodies in which the V_{H} and V_{L} domains are expressed on a single polypeptide chain, but using a linker that is too short to allow for pairing between the two domains on the same chain, thereby forcing the domains to pair with complementary domains of another chain and creating two antigen-binding sites (see e.g., Holliger, P., et al. (1993) Proc. Natl. Acad. Sci. USA 90:6444-6448; Poijak, R. J., et al. (1994) Structure 2:1121-1123).

A bispecific antibody can be formed of an antigen-binding region specific for PSMA and an antigen-binding region specific for an effector cell which has tumoricidal or tumor inhibitory activity. The two antigen-binding regions of the bispecific antibody are either chemically linked or can be expressed by a cell genetically engineered to produce the bispecific antibody. (See generally, Fanger et al., 1995 Drug News & Perspec. 8(3):133-137). Suitable effector cells having tumoricidal activity include but are not limited to cytotoxic T-cells (primarily CD8⁺ cells), natural killer cells, etc. An effective amount of a bispecific antibody according to the invention can be administered to a subject with cancer and the bispecific antibody kills and/or inhibits proliferation of the cancer cells after localization at sites of primary or metastatic tumors bearing PSMA.

In certain embodiments, the antibodies of the ADCs, or from which the antigen-binding fragments of the ADCs are derived, are human antibodies. The term “human antibody”, as used herein, is intended to include antibodies having variable and constant regions derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse have been grafted onto human framework sequences (referred to herein as “humanized antibodies”). Human antibodies directed against PSMA can be generated using transgenic mice carrying parts of the human immune system rather than the mouse system. Some examples of which were described above.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and therefore will not provoke human anti-mouse antibody (HAMA) responses when administered to humans. In general, but not intended to be limiting, the mice are 6-16 weeks of age upon the first immunization. For example, a purified or enriched preparation of PSMA antigen (e.g., recombinant PSMA or PSMA-expressing cells) is used to immunize the mice intraperitoneally (IP), although other routes of immunization known to one of ordinary skill in the art are also possible. PSMA antigen is injected in combination with an adjuvant, such as complete Freund's adjuvant, and, in some embodiments, the initial injection is followed by booster immunizations with antigen in an adjuvant, such as incomplete Freund's adjuvant. The immune response is monitored over the course of the immunization protocol with plasma samples obtained by, for example, retroorbital bleeds. The plasma is screened by ELISA, and mice with sufficient titers of anti-PSMA human immunoglobulin are used for fusions. Mice are boosted intravenously with antigen 3 days before sacrifice and removal of the spleen.

The antibody or antigen-binding fragment thereof of the ADCs can, in some embodiments, be selected for the ability to bind live PSMA-expressing cells. In order to demonstrate binding to live PSMA-expressing cells, flow cytometry can be used. For example, PSMA-expressing cells lines (grown under standard growth conditions) or prostate cancer cells that express PSMA are mixed with various concentrations of monoclonal antibodies in PBS containing 0.1% Tween 80 and 20% mouse serum, and incubated at 37° C. for 1 hour. After washing, the cells are reacted with fluorescein-labeled anti-human IgG secondary antibody (if human anti-PSMA antibodies were used) under the same conditions as the primary antibody staining. The samples can be analyzed by a fluorescence activated cell sorter (FACS) instrument using light and side scatter properties to gate on single cells. An alternative assay using fluorescence microscopy can be used (in addition to or instead of) the flow cytometry assay. Cells can be stained and examined by fluorescence microscopy. This method allows visualization of individual cells, but may have diminished sensitivity depending on the density of the antigen. It follows, that the ADCs, in some embodiments, bind live cells. The ADCs, in some embodiments, therefore, do not require cell lysis to bind PSMA.

The antibodies can, in some embodiments, promote cytolysis of PSMA-expressing cells. Cytolysis can be complement-mediated or can be mediated by effector cells. In one embodiment, the cytolysis is carried out in a living organism, such as a mammal, and the live cell is a tumor cell. Examples of tumors which can be targeted with the antibodies or antigen-binding fragments thereof include, any tumor that expresses PSMA (this includes tumors with neovasculature expressing PSMA), such as, prostate, bladder, pancreas, lung, colon, kidney, melanomas and sarcomas. In one embodiment, the tumor cell is a prostate cancer cell.

The testing of cytolytic activity in vitro by chromium release assay can provide an initial screening prior to testing in vivo models. This testing can be carried out using standard chromium release assays. Briefly, polymorphonuclear cells (PMN), or other effector cells, from healthy donors can be purified by Ficoll Hypaque density centrifugation, followed by lysis of contaminating erythrocytes. Washed PMNs can be suspended in RPMI supplemented with 10% heat-inactivated fetal calf serum and mixed with ⁵¹Cr labeled cells expressing PSMA, at various ratios of effector cells to tumor cells (effector cells:tumor cells). Purified anti-PSMA IgGs can then be added at various concentrations. Irrelevant IgG can be used as a negative control. Assays can be carried out for 0-120 minutes at 37° C. Samples can be assayed for cytolysis by measuring ⁵¹Cr release into the culture supernatant. Anti-PSMA monoclonal antibodies and/or ADCs can also be tested in combinations with each other to determine whether cytolysis is enhanced with multiple monoclonal antibodies and/or ADCs. Antibodies that bind to PSMA and/or ADCs also can be tested in an in vivo model (e.g., in mice) to determine their efficacy in mediating cytolysis and killing of cells expressing PSMA, e.g., tumor cells.

The antibodies of the ADCs, or from which the antigen-binding fragments of the ADCs are derived, can be selected, for example, based on the following criteria, which are not intended to be exclusive:
1) binding to live cells expressing PSMA;
2) high affinity of binding to PSMA;
3) binding to a unique epitope on PSMA (i.e., an epitope not recognized by a previously produced antibody);
4) opsonization of cells expressing PSMA;
5) mediation of growth inhibition, phagocytosis and/or killing of cells expressing PSMA in the presence of effector cells;
6) modulation (inhibition or enhancement) of NAALADase, folate hydrolase, dipeptidyl peptidase IV and/or γ-glutamyl hydrolase activities;
7) growth inhibition, cell cycle arrest and/or cytotoxicity in the absence of effector cells;
8) internalization of PSMA;
9) binding to a conformational epitope on PSMA;
10) minimal cross-reactivity with cells or tissues that do not express PSMA; and
11) preferential binding to dimeric forms of PSMA rather than monomeric forms of PSMA.
   The antibodies can meet one or more, and possibly all, of these criteria.

In one embodiment, the antibody or antigen-binding fragment thereof binds to a conformational epitope, such as a conformational epitope within the extracellular domain of PSMA. To determine if an anti-PSMA antibody or antigen-binding fragment thereof binds to conformational epitopes, each antibody can be tested in assays using native protein (e.g., non-denaturing immunoprecipitation, flow cytometric analysis of cell surface binding) and denatured protein (e.g., Western blot, immunoprecipitation of denatured proteins). A comparison of the results will indicate whether the antibody or antigen-binding fragment thereof binds a conformational epitope. Antibodies or antigen-binding fragments thereof that bind to native protein but not denatured protein are, in some embodiments, those that bind conformational epitopes. It follows, that the ADCs, in some embodiments, bind conformational epitopes of PSMA.

In another embodiment, the antibody or antigen-binding fragment thereof binds to a dimer-specific epitope on PSMA. Generally, antibodies or antigen-binding fragments thereof which bind to a dimer-specific epitope preferentially bind the PSMA dimer rather than the PSMA monomer. To determine if an antibody or antigen-binding fragment thereof binds preferentially (i.e., selectively and/or specifically) to a PSMA dimer, the antibody or antigen-binding fragment thereof can be tested in assays (e.g., immunoprecipitation followed by Western blotting) using native dimeric PSMA protein and dissociated monomeric PSMA protein. A comparison of the results will indicate whether the antibody or antigen-binding fragment thereof binds preferentially to the dimer. In some embodiments, the antibodies or antigen-binding fragments thereof bind to the PSMA dimer but not to the monomeric PSMA protein. It follows, that the ADCs, in some embodiments, bind to a dimer-specific epitope on PSMA.

The invention, therefore, also includes ADCs that selectively bind PSMA multimers. As used herein, particularly with respect to the binding of PSMA multimers by the ADCs, “selectively binds” means that an antibody preferentially binds to a PSMA protein multimer (e.g., with greater avidity, greater binding affinity) rather than to a PSMA protein monomer. In some embodiments, the ADCs of the invention bind to a PSMA protein multimer with an avidity and/or binding affinity that is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 7-fold, 10-fold, 20-fold, 30-fold, 40-fold, 50-fold, 70-fold, 100-fold, 200-fold, 300-fold, 500-fold, 1000-fold or more than that exhibited by the ADC for a PSMA protein monomer. The ADC can, in some embodiments, selectively bind a PSMA protein multimer, and not a PSMA protein monomer, i.e., exclusively binds to a PSMA protein multimer. In some embodiments, the ADC selectively binds a PSMA protein dimer.

A PSMA protein multimer, as used herein, is a protein complex of at least two PSMA proteins or fragments thereof. The PSMA protein multimers can be composed of various combinations of full-length PSMA proteins (e.g., SEQ ID NO: 1), recombinant soluble PSMA (rsPSMA, e.g., amino acids 44-750 of SEQ ID NO: 1) and fragments of the foregoing that form multimers (i.e., that retain the protein domain required for forming dimers and/or higher order multimers of PSMA). In some embodiments, at least one of the PSMA proteins forming the multimer is a recombinant, soluble PSMA (rsPSMA) polypeptide. The PSMA protein multimers can be dimers, such as those formed from recombinant soluble PSMA protein. In one embodiment, the dimer is a rsPSMA homodimer. The PSMA protein multimers referred to herein are believed to assume a native conformation and can have such a conformation. The PSMA proteins in certain embodiments are noncovalently bound together to form the PSMA protein multimer. For example, it has been discovered that PSMA protein noncovalently associates to form dimers under non-denaturing conditions. The PSMA protein multimers can retain the activities of PSMA. The PSMA activity may be an enzymatic activity, such as folate hydrolase activity, NAALADase activity, dipeptidyl peptidase IV activity or γ-glutamyl hydrolase activity. Methods for testing the PSMA activity of multimers are well known in the art (reviewed by O'Keefe et al. in: Prostate Cancer: Biology, Genetics, and the New Therapeutics, L. W. K. Chung, W. B. Isaacs and J. W. Simons (eds.) Humana Press, Totowa, N.J., 2000, pp. 307-326).

The antibody or antigen-binding fragment thereof of the ADCs can bind to and is internalized with PSMA expressed on cells. The mechanism by which the antibody or antigen-binding fragment thereof is internalized with PSMA is not critical to the practice of the present invention. For example, the antibody or antigen-binding fragment thereof can induce internalization of PSMA. Alternatively, internalization of the antibody or antigen-binding fragment thereof can be the result of routine internalization of PSMA. It follows that the ADC can be internalized with PSMA expressed on cells.

The antibodies or antigen-binding fragments thereof, and therefore the ADCs of the invention, can specifically bind cell-surface PSMA and/or rsPSMA with sub-nanomolar affinity. The binding affinities can be about 1×10⁻⁹M or less, about 1×10⁻¹⁰M or less, or about 1×10⁻¹¹M or less. In a particular embodiment the binding affinity is less than about 5×10⁻¹⁰ M.

The antibodies or antigen-binding fragments thereof can, in some embodiments, modulate at least one enzymatic activity of PSMA. The activity can be selected from the group consisting of N-acetylated α-linked acidic dipeptidase (NAALADase), folate hydrolase, dipeptidyl dipeptidase IV, γ-glutamyl hydrolase activity and combinations thereof in vitro or in vivo. The modulation may be enhancement or inhibition of at least one enzymatic activity of PSMA.

Tissue levels of NAALADase can be determined by detergent solubilizing homogenizing tissues, pelleting the insoluble material by centrifugation and measuring the NAALADase activity in the remaining supernatant. Likewise, the NAALADase activity in bodily fluids can also be measured by first pelleting the cellular material by centrifugation and performing a typical enzyme assay for NAALADase activity on the supernatant. NAALADase enzyme assays have been described by Frieden, 1959, J. Biol, Chem., 234:2891. In this assay, the reaction product of the NAALADase enzyme is glutamic acid. This is derived from the enzyme catalyzed cleavage of N-acetylaspartylglutamate to yield N-acetylaspartic acid and glutamic acid. Glutamic acid, in a NAD(P)⁺ requiring step, yields 2-oxoglutarate plus NAD(P)H in a reaction catalyzed by glutamate dehydrogenase. Progress of the reaction can easily and conveniently be measured by the change in absorbance at 340 nm due to the conversion of NAD(P)⁺ to NAD(P)H.

Folate hydrolase activity of PSMA can be measured by performing enzyme assays as described by Heston and others (e.g., Clin. Cancer Res. 2(9):1445-51, 1996; Urology 49(3A Suppl):104-12, 1997). Folate hydrolases such as PSMA remove the gamma-linked glutamates from polyglutamated folates. Folate hydrolase activity can be measured using substrates such as methotrexate tri-gamma glutamate (MTXGlu3), methotrexate di-gamma glutamate (MTXGlu2) or pteroylpentaglutamate (PteGlu5), for example using capillary electrophoresis (see Clin. Cancer Res. 2(9):1445-51, 1996). Timed incubations of PSMA with polyglutamated substrates is followed by separation and detection of hydrolysis products.

An ADC of the invention comprises an antibody or antigen-binding fragment thereof conjugated to MMAE or MMAF. The antibody or antigen-binding fragment thereof can be, in some embodiments, conjugated to MMAE or MMAF with a compound of the following formula (Formula 1): -Aₙ-Yₘ—Zₘ—Xₙ—Wₙ—, wherein A is a carboxylic acyl unit; Y is an amino acid; Z is an amino acid; X and W are each a self-immolative spacer; n is an integer of 0 or 1; and m is an integer of 0 or 1, 2, 3, 4, 5 or 6. A conjugate of the present invention, in some embodiments, is represented by the formula (Formula 2): L-{Aₙ-Yₘ—Zₘ—Xₙ—Wₙ-D}ₚ wherein L is an antibody or antigen-binding fragment thereof that binds PSMA, D is MMAE or MMAF and p is an integer of 1, 2, 3, 4, 5, 6, 7 or 8. The other components are as described above. In one embodiment, the carboxylic unit “Aₙ” is linked to the antibody or antigen-binding fragment via a sulfur atom derived from the antibody or antigen-binding fragment:

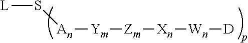

In one embodiment, A is

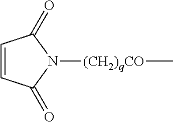

in which q is 1-10. Therefore, in one embodiment, the conjugate is:

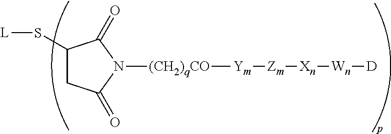

wherein L, Y, Z, X, W, D, n, m, q and p are as previously defined.

In another embodiment, A is 4-(N-succinimidomethyl)cyclohexane-1-carbonyl, m-succinimidobenzoyl, 4-(p-succinimidophenyl)-butyryl, 4-(2-acetamido)benzoyl, 3-thiopropionyl, 4-(1-thioethyl)-benzoyl, 6-(3-thiopropionylamido)-hexanoyl or maleimide caproyl. In a further embodiment, A is maleimide caproyl. Representative examples of various carboxylic acyl units and methods for their synthesis and attachment are described in U.S. Pat. No. 6,214,345, the entire contents of which are herein incorporated by reference.

In another embodiment, Y is alanine, valine, leucine, isoleucine, methionine, phenylalanine, tryptophan or proline. In yet another embodiment, Y is valine. In a further embodiment, Z is lysine, lysine protected with acetyl or formyl, arginine, arginine protected with tosyl or nitro groups, histidine, ornithine, ornithine protected with acetyl or formyl, or citrulline. In still a further embodiment, Z is citrulline. In one embodiment Yₘ—Zₘ is valine-citrulline. In another embodiment, Yₘ—Zₘ is a protein sequence which is selectively cleavable by a protease.

In a further embodiment, X is a compound having the formula

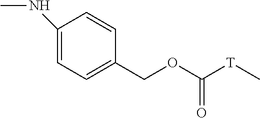

in which T is O, N, or S. In another embodiment, X is a compound having the formula —HN—R¹—COT in which R¹ is C₁-C₅ alkyl, T is O, N or S. In a further embodiment, X is a compound having the formula

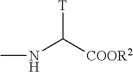

in which T is O, N, or S, R² is H or C₁-C₅ alkyl. In one embodiment, X is p-aminobenzylcarbamoyloxy. In another embodiment, X is p-aminobenzylalcohol. In a further embodiment, X is p-aminobenzylcarbamate. In yet a further embodiment, X is p-aminobenzyloxycarbonyl. In another embodiment, X is γ-aminobutyric acid; α,α-dimethyl γ-aminobutyric acid or β,β-dimethyl γ-aminobutyric acid.

In some embodiments, W is

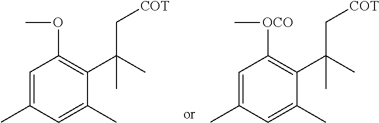

in which T is O, S or N.

In one embodiment, the compound of Formula 1 is maleimidocaproyl. Maleimidocaproyl has been used for conjugation of two specific auristatins to an anti-CD30 mAb (AC10) (Doronina, Svetlana et al. “Novel Linkers for Monoclonal Antibody-Mediated Delivery of Anticancer Agents”, AACR, Anaheim, Calif., Abstract No. 1421, Apr. 16-20, 2005). Maleimidocaproyl reacts with thiol groups to form a thioether.

MMAE or MMAF can be conjugated to an antibody or antigen-binding fragment thereof using methods known to those of ordinary skill in the art (e.g., See, Niemeyer, C M, Bioconjugation Protocols, Strategies and Methods, Humana Press, 2004) or as described herein. In some embodiments, more than one MMAE or MMAF molecule is conjugated to the antibody or antigen-binding fragment thereof. In other embodiments, 1, 2, 3, 4, 5, 6, 7 or 8 MMAE or MMAF molecules are conjugated to the antibody or antigen-binding fragment thereof. In still other embodiments, at least 3, 4 or 5 MMAE or MMAF molecules are conjugated to the antibody or antigen-binding fragment thereof. In further embodiments, 3, 4 or 5 MMAE or MMAF molecules are conjugated to the antibody or antigen-binding fragment thereof.

The ADCs of the invention have been found to have particularly high levels of selectivity when killing of non-PSMA-expressing cells is compared to killing of PSMA-expressing cells. Therefore, in some embodiments, the ADCs have a PC-3™ cell to C4-2 cell or LNCaP™ cell selectivity of at least 250. In other embodiments, the selectivity is at least 300, 350, 400, 450, 500, 600, 700, 800, 900, 1000, 1100, 1200, 1300, 1400, 1500, 1600, 1700, 1800, 1900, 2000, 2250, 2500, 2750, 3000, 3500, 4000, 4500, 5000, 5500, 6000, 6500, 7000, 7500, 8000, 8500, 9000, 9500, 10000, 11000, 12000, 13000, 14000, 15000, 17500, 20000 or more. In some embodiments, the selectivity is between 250-500, 500-750, 750-1000, 1000-2000, 2000-5000, 5000-10000, 10000-15000 or 15000-20000. “Selectivity”, as defined herein, refers to the ratio of IC₅₀ values of an ADC on PC-3™ cells (non-PSMA-expressing cells) to C4-2 cells or LNCaP™ cells (PSMA-expressing cells).

It has also been found that the ADCs of the invention mediate, in some embodiments, PSMA-expressing specific cell killing at very low concentrations, such as at or near picomolar concentrations. The ADCs, in some embodiments, exhibit IC₅₀s at concentrations of less than about 1×10⁻¹⁰M, less than about 1×10⁻¹¹M, or less than about 1×10⁻¹²M. In a particular embodiment, an IC₅₀ is achieved at a concentration of less than about 1.5×10⁻¹¹M. In another embodiment, the ADCs provided exhibit IC₅₀s of between 10-210, 40-210, 60-210 or 65-210 pM. In yet another embodiment, the ADCs provided exhibit IC₅₀s of about 10, 40, 60 or 80 pM. In still another embodiment, the ADCs provided exhibit IC₅₀s of about 11, 42, 60 or 83 pM.

It has also been found that the ADCs, in some embodiments, effect a cure rate in mice of at least 20%, 30%, 40%, 50%, 60%, 70%, 80%, 90% or 95%. In other embodiments, the cure rate in mice is about 20%, 30%, 40%, 50%, 60%, 70%, 80%, 90% or 95%. In still other embodiments, the cure rate is 20-40%, 40-60% or 60-80%. As used herein, “cure rate” refers to the number of mice still alive after about 500 days from the start of a study period, with no evidence of a tumor and no measurable PSA levels, divided by the number of mice at the beginning of the study period. To assess the cure rate, mice are administered 6 mg/kg ADC with a regimen of q4d×6. In some embodiments, the number of mice at the beginning of the study is at least 5, 6, 7, 8, 9, 10, 12, 15, 17, 20, 25, 30 or more mice. Further details regarding an example of such a study are provided herein below in the Examples. In one embodiment, the mice are those that are a model of androgen-independent human prostate cancer. In another embodiment, the mice are nude mice engrafted with C4-2 cells intramuscularly in the left hind-leg. Techniques for determining the presence of a tumor and for measuring PSA levels are well known to those of ordinary skill in the art.

Binding of the ADCs of the invention to live PSMA-expressing cells can inhibit the growth of PSMA-expressing cells, result in cell-cycle arrest (e.g., G₂/M arrest), promote apoptosis of PSMA-expressing cells, etc. As used herein, “result in cell-cycle arrest” refers to an increase in the number of cells in the G₂/M phase due to the administration of an ADC. In some embodiments, the ADCs can effect apoptosis. In other embodiments, the ADCs result in both cell cycle arrest and subsequent apoptosis. The ADCs of the invention, therefore, can be used in various in vitro and in vivo methods for effecting these possible endpoints. In particular, the ADCs of the invention can be used in methods for treating PSMA-mediated disease.

As used herein, a “PSMA-mediated disease” is any disease in which PSMA is causative or a symptom of the disease. PSMA-mediated diseases also include diseases or disorders in which there is aberrant (e.g., overexpression) of PSMA. PSMA is a 100 kD Type II membrane glycoprotein expressed in prostate tissues (Horoszewicz et al., 1987, Anticancer Res. 7:927-935; U.S. Pat. No. 5,162,504). PSMA was characterized as a type II transmembrane protein having sequence identity with the transferrin receptor (Israeli et al., 1994, Cancer Res. 54:1807-1811) and with NAALADase activity (Carter et al., 1996, Proc. Natl. Acad. Sci. U.S.A. 93:749-753). More importantly, PSMA is expressed in increased amounts in prostate cancer, and elevated levels of PSMA are also detectable in the sera of these patients (Horoszewicz et al., 1987; Rochon et al., 1994, Prostate 25:219-223; Murphy et al., 1995, Prostate 26:164-168; and Murphy et al., 1995, Anticancer Res. 15:1473-1479). Therefore, a PSMA-mediated disorder is, for example, prostate cancer. PSMA expression increases with disease progression, becoming highest in metastatic, hormone-refractory disease for which there is no present therapy. In addition, provocative data indicates that PSMA is also abundantly expressed on the neovasculature of a variety of other important tumors, including bladder, pancreas, sarcoma, melanoma, lung, and kidney tumor cells, but not on normal vasculature. PSMA-mediated diseases, therefore, include cancers in which PSMA is expressed on the cells of the tumor or of the tumor neovasculature.

Compositions and methods are, therefore, provided that can be used to treat any PSMA-mediated disorder. For example, ADCs can be used to inhibit the neovascularization of a tumor. In another example, PSMA ADCs can be used to kill tumor cells. In some embodiments, two or more different ADCs are used in combination. In another embodiment, one or more unconjugated anti-PSMA antibodies or antigen-binding fragments thereof can be combined with one or more ADCs in a single therapy to achieve a desired therapeutic effect. As an illustration, an unconjugated anti-PSMA antibody that mediates highly effective killing of target cells in the presence of effector cells and/or that inhibits the growth of cells expressing PSMA can be used with one or more ADCs. In yet another embodiment, the ADCs can be combined with one or more additional therapeutic agents. Such therapeutic agents include antitumor agents, such as docetaxel; corticosteroids, such as prednisone or hydrocortisone; immunostimulatory agents; immunomodulators; or some combination thereof.

Antitumor agents include cytotoxic agents, chemotherapeutic agents and agents that act on tumor neovasculature. Cytotoxic agents include cytotoxic radionuclides, chemical toxins and protein toxins. The cytotoxic radionuclide or radiotherapeutic isotope can be an alpha-emitting isotope such as ²²⁵Ac, ²¹¹At, ²¹²Bi, ²¹³Bi, ²¹²Pb, ²²⁴Ra or ²²³Ra. Alternatively, the cytotoxic radionuclide can be a beta-emitting isotope such as ¹⁸⁶Rh, ¹⁸⁸Rh, ¹⁷⁷Lu, ⁹⁰Y, ¹³¹I, ⁶⁷Cu, ⁶⁴Cu, ¹⁵³Sm or ¹⁶⁶Ho. Further, the cytotoxic radionuclide can emit Auger and low energy electrons and include the isotopes ¹²⁵I, ¹²³I or ⁷⁷Br.

Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Other antineoplastic agents include dolastatins (U.S. Pat. Nos. 6,034,065 and 6,239,104) and derivatives thereof. Dolastatins and derivatives thereof include dolastatin 10 (dolavaline-valine-dolaisoleuine-dolaproine-dolaphenine) and the derivatives auristatin PHE (dolavaline-valine-dolaisoleuine-dolaproine-phenylalanine-methyl ester) (Pettit, G. R. et al., Anticancer Drug Des. 13(4):243-277, 1998; Woyke, T. et al., Antimicrob. Agents Chemother. 45(12):3580-3584, 2001), and aurastatin E and the like. Toxins also include poisonous lectins, plant toxins such as ricin, abrin, modeccin, botulina and diphtheria toxins. Other chemotherapeutic agents are known to those skilled in the art.

Agents that act on the tumor vasculature include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein) and interferon inducible protein 10 (U.S. Pat. No. 5,994,292). A number of other antiangiogenic agents are also contemplated and include: 2ME2, Angiostatin, Angiozyme, Anti-VEGF RhuMAb, Apra (CT-2584), Avicine, Benefin, BMS275291, Carboxyamidotriazole, CC4047, CC5013, CC7085, CDCl801, CGP-41251 (PKC 412), CM101, Combretastatin A-4 Prodrug, EMD 121974, Endostatin, Flavopiridol, Genistein (GCP), Green Tea Extract, IM-862, ImmTher, Interferon alpha, Interleukin-12, Iressa (ZD1839), Marimastat, Metastat (Col-3), Neovastat, Octreotide, Paclitaxel, Penicillamine, Photofrin, Photopoint, PI-88, Prinomastat (AG-3340), PTK787 (ZK22584), R0317453, Solimastat, Squalamine, SU 101, SU 5416, SU-6668, Suradista (FCE 26644), Suramin (Metaret), Tetrathiomolybdate, Thalidomide, TNP-470 and Vitaxin. Additional antiangiogenic agents are described by Kerbel, J. Clin. Oncol. 19(18s):45s-51s, 2001, which is incorporated by reference herein.

The ADCs can be administered with one or more immunostimulatory agents to induce or enhance an immune response, such as IL-2 and immunostimulatory oligonucleotides (e.g., those containing CpG motifs). Immunostimulatory agents can, in some embodiments, stimulate specific arms of the immune system, such as natural killer (NK) cells that mediate antibody-dependent cell cytotoxicity (ADCC). Immunostimulatory agents include interleukin-2, α-interferon, γ-interferon, tumor necrosis factor alpha (TNFα), immunostimulatory oligonucleotides or a combination thereof. Immunomodulators include cytokines, chemokines, adjuvants or a combination thereof. Chemokines useful in increasing immune responses include but are not limited to SLC, ELC, MIP3α, MIP3β, IP-10, MIG, and combinations thereof.

The other therapeutic agent can also be a vaccine. In some embodiments, the vaccine immunizes a subject against PSMA. Such vaccines, in some embodiments, include antigens, such as PSMA dimers, with, optionally, one or more adjuvants to induce or enhance an immune response. An adjuvant is a substance which potentiates the immune response. Adjuvants of many kinds are well known in the art. Specific examples of adjuvants include monophosphoryl lipid A (MPL, SmithKline Beecham); saponins including QS21 (SmithKline Beecham); immunostimulatory oligonucleotides (e.g., CpG oligonucleotides described by Kreig et al., Nature 374:546-9, 1995); incomplete Freund's adjuvant; complete Freund's adjuvant; montanide; vitamin E and various water-in-oil emulsions prepared from biodegradable oils such as squalene and/or tocopherol, Quil A, Ribi Detox, CRL-1005, L-121, and combinations thereof. Formulations, such as those described in U.S. application Ser. No. 10/976,352, are also contemplated for use as vaccines in the methods provided herein. The disclosure of such formulations are incorporated herein by reference.

The vaccines can, in some embodiments, include one or more of the isolated PSMA protein multimers described herein, such as the PSMA protein dimer. In some embodiments, a PSMA protein multimer composition contains at least about 10% PSMA protein multimer (of the total amount of PSMA protein in the composition). In other embodiments, the PSMA protein multimer composition contains at least about 20%, 30%, 40%, 50%, 60%, 70%, 75%, 80%, 85%, 90%, 95%, 99% or 99.5% PSMA protein multimer. In one embodiment, the PSMA protein multimer composition contains substantially pure PSMA protein multimer, with substantially no PSMA protein monomer. It is understood that the list of specific percentages includes by inference all of the unnamed percentages between the recited percentages.

Cytokines can also be used in vaccination protocols as a result of their lymphocyte regulatory properties. Many cytokines useful for such purposes will be known to one of ordinary skill in the art, including interleukin-2 (IL-2); IL-4; IL-5; IL-12, which has been shown to enhance the protective effects of vaccines (see, e.g., Science 268: 1432-1434, 1995); GM-CSF; IL-15; IL-18; combinations thereof, and the like. Thus cytokines can be administered in conjunction with antigen, chemokines and/or adjuvants to increase an immune response.

The other therapeutic agents can be present in the compositions of the invention or used in the methods of the invention in unconjugated form or in conjugated form, such as conjugated to an anti-PSMA antibody or antigen-binding fragment thereof. Coupling of one or more toxin molecules to the anti-PSMA antibody or antigen-binding fragment thereof can include many chemical mechanisms, for instance covalent binding, affinity binding, intercalation, coordinate binding and complexation.

The covalent binding can be achieved either by direct condensation of existing side chains or by the incorporation of external bridging molecules. Many bivalent or polyvalent agents are useful in coupling protein molecules to other proteins, peptides or amine functions, etc. For example, the literature is replete with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents.

In some embodiments, it is contemplated that one may wish to first derivatize the antibody, and then attach the therapeutic agent to the derivatized product. Suitable cross-linking agents for use in this manner include, for example, SPDP (N-succinimidyl-3-(2-pyridyldithio)propionate), and SMPT, 4-succinimidyl-oxycarbonyl-methyl-(2-pyridyldithio)toluene.

In addition, protein toxins can be fused to the anti-PSMA antibody or antigen-binding fragment thereof by genetic methods to form a hybrid immunotoxin fusion protein. The fusion proteins can include additional peptide sequences, such as peptide spacers which operatively attach, for example, the anti-PSMA antibody and toxin, as long as such additional sequences do not appreciably affect the targeting or toxin activities of the fusion protein. The proteins can be attached by a peptide linker or spacer, such as a glycine-serine spacer peptide, or a peptide hinge, as is well known in the art. Thus, for example, the C-terminus of an anti-PSMA antibody or antigen-binding fragment thereof can be fused to the N-terminus of the protein toxin molecule to form an immunotoxin that retains the binding properties of the anti-PSMA antibody. Other fusion arrangements will be known to one of ordinary skill in the art. To express the fusion immunotoxin, the nucleic acid encoding the fusion protein is inserted into an expression vector in accordance with standard methods, for stable expression of the fusion protein, such as in mammalian cells, such as CHO cells. The fusion protein can be isolated and purified from the cells or culture supernatant using standard methodology, such as a PSMA affinity column.

Radionuclides typically are coupled to an antibody or antigen-binding fragment thereof by chelation. For example, in the case of metallic radionuclides, a bifunctional chelator is commonly used to link the isotope to the antibody or other protein of interest. Typically, the chelator is first attached to the antibody, and the chelator-antibody conjugate is contacted with the metallic radioisotope. A number of bifunctional chelators have been developed for this purpose, including the diethylenetriamine pentaacetic acid (DTPA) series of amino acids described in U.S. Pat. Nos. 5,124,471, 5,286,850 and 5,434,287, which are incorporated herein by reference. As another example, hydroxamic acid-based bifunctional chelating agents are described in U.S. Pat. No. 5,756,825, the contents of which are incorporated herein. Another example is the chelating agent termed p-SCN-Bz-HEHA (1,4,7,10,13,16-hexaazacyclo-octadecane-N,N′,N″,N′″,N″″,N′″″-hexaacetic acid) (Deal et al., J. Med. Chem. 42:2988, 1999), which is an effective chelator of radiometals such as ²²⁵Ac. Yet another example is DOTA (1,4,7,10-tetraazacyclododecane N,N′,N″,N′″-tetraacetic acid), which is a bifunctional chelating agent (see McDevitt et al., Science 294:1537-1540, 2001) that can be used in a two-step method for labeling followed by conjugation.

Other therapeutic agents also include replication-selective viruses. Replication-competent virus such as the p53 pathway targeting adenovirus mutant dl1520, ONYX-015, kills tumor cells selectively (Biederer, C. et al., J. Mol. Med. 80(3):163-175, 2002). The virus can, in some embodiments, be conjugated to PSMA antibodies or antigen-binding fragments thereof.

The compositions provided of the present invention can be used in conjunction with other therapeutic treatment modalities. Such other treatments include surgery, radiation, cryosurgery, thermotherapy, hormone treatment, chemotherapy, vaccines and other immunotherapies.

The ADCs of the invention, such as through their antibody or antigen-binding fragment thereof, can be linked to a label. Labels include, for example, fluorescent labels, enzyme labels, radioactive labels, nuclear magnetic resonance active labels, luminescent labels or chromophore labels.

The compositions provided can include a physiologically or pharmaceutically acceptable carrier, excipient or stabilizer mixed with the ADC. In some embodiments, when a composition comprises two or more different ADCs, each of the antibodies or antigen-binding fragments thereof of the ADCs binds to a distinct conformational epitope of PSMA.

As used herein, “target cell” shall mean any undesirable cell in a subject (e.g., a human or animal) that can be targeted by an ADC of the invention. In some embodiments, the target cell is a cell expressing or overexpressing PSMA. Cells expressing PSMA or PSMA-expressing cells, typically include tumor cells, such as prostate, bladder, pancreas, lung, kidney, colon tumor cells, as well as melanoma and sarcoma cells.

Pharmaceutical compositions of the invention can be administered in combination therapy, i.e., combined with other agents. For example, the combination therapy can include a composition of the present invention with at least one anti-tumor agent, immunomodulator, immunostimulatory agent or other conventional therapy. The other agent can be conjugated to or formed as a recombinant fusion molecule with a PSMA antibody or antigen-binding fragment thereof for directed targeting of the agent to PSMA-expressing cells. In another embodiment the other therapeutic agent can be unconjugated. Additional therapeutic agents can be administered or contacted with the PSMA-expressing cells through co-administration. “Co-administering,” as used herein, refers to administering two or more therapeutic agents simultaneously as an admixture in a single composition, or sequentially, and close enough in time so that the compounds may exert an additive or even synergistic effect. In still other embodiments, an additional therapeutic agent can be administered before, during or after the administration of one or more ADCs or compositions thereof.

As used herein, “pharmaceutically acceptable carrier” or “physiologically acceptable carrier” includes any and all salts, solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active compound, can be coated in a material to protect the compound from the action of acids and other natural conditions that may inactivate the compound.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents, such as supplementary immune potentiating agents including adjuvants, chemokines and cytokines. When used in medicine, the salts should be pharmaceutically acceptable, but non-pharmaceutically acceptable salts may conveniently be used to prepare pharmaceutically-acceptable salts thereof and are not excluded from the scope of the invention.

A salt retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

An ADC can be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation of the compounds, which is, in some embodiments, isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulations suitable for oral, subcutaneous, intravenous, intramuscular, etc. administration can be found in Remington's Pharmaceutical Sciences, Mack Publishing Co., Easton, Pa.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The therapeutics of the invention can be administered by any conventional route, including injection or by gradual infusion over time. The administration may, for example, be oral, intravenous, intraperitoneal, intramuscular, intracavity, intratumor, or transdermal. When compounds containing antibodies are used therapeutically, routes of administration include intravenous and by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp. 1694-1712; incorporated by reference). Those of skill in the art can readily determine the various parameters and conditions for producing antibody aerosols without resorting to undue experimentation.

The compositions of the invention are administered in effective amounts. An “effective amount” is that amount of any of the ADCs provided herein that alone, or together with further doses and/or other therapeutic agents, produces the desired response, e.g., treats a PSMA-mediated disease in a subject. This can involve only slowing the progression of the disease temporarily, although in some embodiments, it involves halting the progression of the disease permanently. This can be monitored by routine methods. The desired response to treatment of the disease or condition also can be delaying the onset or even preventing the onset of the disease or condition. An amount that is effective can be the amount of an ADC alone which produces the desired therapeutic endpoint. An amount that is effective is also the amount of an ADC in combination with another agent that produces the desired result.

Such amounts will depend, of course, on the particular PSMA-mediated disease being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of an ADC, alone or in combination with another agent, for producing the desired response in a unit of weight or volume suitable for administration to a patient. The response can, for example, be measured by determining the physiological effects of the ADC composition, such as regression of a tumor or decrease of disease symptoms. Other assays will be known to one of ordinary skill in the art and can be employed for measuring the level of the response.

The doses of ADCs administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses can range from about 10 μg/kg to about 100,000 μg/kg. In some embodiments, the doses can range from about 0.1 mg/kg to about 20 mg/kg. In still other embodiments, the doses range from about 0.1 mg/kg to 5 mg/kg, 0.1 mg/kg to 10 mg/kg or 0.1 mg/kg to 15 mg/kg. In yet other embodiments, the doses range from about 1 mg/kg to 5 mg/kg, 5 mg/kg to 10 mg/kg, 10 mg/kg to 15 mg/kg or 15 mg/kg to 20 mg/kg. In further embodiments, the dose is about 0.1 mg/kg, 0.5 mg/kg, 1 mg/kg, 2 mg/kg, 3 mg/kg, 5 mg/kg, 7 mg/kg, 10 mg/kg, 12 mg/kg, 15 mg/kg, 17 mg/kg, 20 mg/kg, 25 mg/kg or 30 mg/kg. In another embodiment, the dose is about 1 mg/kg, 3 mg/kg, 5 mg/kg or 6 mg/kg. Based upon the composition, the dose can be delivered continuously, such as by continuous pump, or at periodic intervals. In some embodiments, when the ADC is administered intravenously, the dose is between 0.1 and 20 mg/kg or any value in between. Desired time intervals of multiple doses of a particular composition can be determined without undue experimentation by one skilled in the art. Other protocols for the administration of the compositions provided will be known to one of ordinary skill in the art, in which the dose amount, schedule of administration, sites of administration, mode of administration and the like vary from the foregoing. In some embodiments, subjects are administered the ADC with a dose regimen of q4d×3 or q4d×6. In one embodiment, the dose is administered intravenously. In another embodiment, the dose regimen is a single intravenous dose.

Administration of ADC compositions to mammals other than humans, e.g. for testing purposes or veterinary therapeutic purposes, is carried out under substantially the same conditions as described above.

The compositions of the present invention have in vitro and in vivo diagnostic and therapeutic utilities. For example, these molecules can be administered to cells in culture, e.g. in vitro or ex vivo, or in a subject, e.g., in vivo, to treat, prevent or diagnose a variety of PSMA-mediated diseases. As used herein, the term “subject” is intended to include humans and non-human animals. Subjects include a human patient having a disorder characterized by expression, typically aberrant expression (e.g., overexpression) of PSMA, such disorders are included in the definition of “PSMA-mediated disease”.

The compositions provided herein can be utilized in in vivo therapy of cancer. The ADCs can be used to inhibit proliferation of the malignant cells or tissues following administration and localization of the conjugates. The compositions provided can include anti-PSMA antibodies, in some embodiments, that may mediate tumor destruction by complement fixation or antibody-dependent cellular cytotoxicity. Alternatively, the compositions can contain an additional therapeutic agent to result in synergistic therapeutic effects (Baslya and Mendelsohn, 1994 Breast Cancer Res. and Treatment 29:127-138).

The compositions of the invention can also be administered together with, in some embodiments, complement and/or unconjugated anti-PSMA antibodies. Accordingly, within the scope of the invention are compositions comprising ADC and serum or complement. These compositions are advantageous in that the complement is located in close proximity to the human antibodies or antigen-binding fragments thereof. Alternatively, the ADCs, antibodies or antigen-binding fragments thereof and/or complement or serum can be administered separately.

Use of the therapy of the present invention has a number of benefits. Since the ADCs preferentially target PSMA e.g., on prostate cancer cells, other tissue can be spared. As a result, treatment with such biological agents is safer, particularly for elderly patients. Treatment according to the present invention is expected to be particularly effective, in some embodiments, because it can direct high levels of ADCs to the bone marrow and lymph nodes where cancer metastases, such as prostate cancer metastases, can predominate. Treatment in accordance with the present invention can be effectively monitored with clinical parameters such as serum prostate specific antigen and/or pathological features of a patient's cancer, including stage, Gleason score, extracapsular, seminal, vesicle or perineural invasion, positive margins, involved lymph nodes, etc. Alternatively, these parameters can be used to indicate when such treatment should be employed.

Also within the scope of the invention are kits comprising the compositions, e.g., one or more ADCs, of the invention and instructions for use. The kits can further contain at least one additional reagent, such as complement, a chemotherapeutic agent, a corticosteroid, or one or more antibodies that bind PSMA. Other kits can also include PSMA multimers. In another embodiment, a kit can comprise a carrier being compartmentalized to receive in close confinement therein one or more container means or series of container means such as test tubes, vials, flasks, bottles, syringes, or the like. A first of said container means or series of container means may contain one or more anti-PSMA antibodies or antigen-binding fragments thereof. A second container means or series of container means can, in some embodiments, contain MMAE or MMAF or the compound of Formula 1 conjugated to MMAE or MMAF. In some embodiments, a third container means or series of container means contain a compound of Formula 1. Kits for use in in vivo tumor localization and therapy method containing the ADCs can be prepared. The components of the kits can be packaged either in aqueous medium or in lyophilized form. The components of the ADC conjugates can be supplied either in fully conjugated form, in the form of intermediates or as separate moieties to be conjugated by the user of the kit.

As used herein with respect to polypeptides, proteins or fragments thereof, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may be, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

The compositions provided herein can be in lyophilized form or provided in an aqueous medium.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1: Potent Antitumor Activity of an Auristatin-Conjugated, Fully Human Monoclonal Antibody to Prostate-Specific Membrane Antigen

### Materials and Methods

### Cell Lines and Antibodies

LNCaP™ (CRL-1740), PC-3™ (CRL-1435), and 3T3™ (CRL-2752) were obtained from American Type Culture Collection (Rockville, Md.). C4-2 cell line, a sub-cell line from LNCaP™, was obtained from The Cleveland Clinic Foundation (Cleveland, Ohio). A 3T3™-PSMA cell line was obtained from Memorial Sloan-Kettering Cancer Center (New York, N.Y.). LNCaP™, C4-2 and PC-3™ were cultured in RPMI 1640 (Life Technologies, Gaithersburg, Md.), and 3T3™ and 3T3™-PSMA were cultured in DMEM (Life Technologies). Culture media were supplemented with 10% fetal bovine serum (Hyclone, Logan, Utah), L-glutamine, penicillin and streptomycin (Life Technologies). C4-2, LNCaP™ and 3T3™-PSMA cells were determined to express PSMA at levels of approximately 2×10⁵, 6×10⁵ and >1×10⁶ copies/cell, respectively, according to published methods (Ma D, et al., Leukemia 2002; 16:60-6.). C4-2 is an androgen-independent subclone of androgen-dependent LNCaP™ cells. PC-3™ is a de-differentiated prostate cancer cell line that does not express PSMA. PSMA mAbs (AB-PG1-XG1-006 (PTA-4403 and PTA-4404) and Abgenix 4.40.2 (PTA-4360)) were produced as described previously in U.S. patent application Ser. No. 10/395,894 and Schulke N et al., PNAS USA, 2003; 100:12590-5, each of which is herein incorporated by reference in its entirety. Abgenix 4.40.2 was used as a control. A fully human PSMA mAb (IgG1,κ) was raised in mice transgenic for the human immunoglobulin gene locus (XenoMice™, Abgenix, Inc., Fremont, Calif.) following immunization with recombinant soluble PSMA and LNCaP cells as previously described (Schulke N et al., PNAS USA, 2003; 100:12590-5).

### PSMA Internalization

mAbs were modified with bifunctional chelates of cyclohexyl-diethylenetriamine pentaacetic acid (CHX-DTPA) obtained from the National Cancer Institute (Bethesda, Md.), and labeled with ¹¹¹In (PerkinElmer, Boston, Mass.) as previously described (Ma D, et al., Leukemia 2002; 16:60-6; Nikula T K, et al., J Nucl. Med 1999; 40:166-76). ¹¹¹In-labeled mAb was determined to be >90% immunoreactive by incubating the radioconjugate with an excess of 3T3™-PSMA cells and measuring the bound fraction according to published methods (Ma D, et al., Leukemia 2002; 16:60-6; Nikula T K, et al., J Nucl. Med 1999; 40:166-76). For internalization analysis, ¹¹¹In-labeled mAb was incubated with 2×10⁵ C4-2 cells at 37° C. in 5% CO₂. At sequential time points, unbound mAb was removed by washing in PBS and cell-surface mAb was eluted using low pH buffer (pH 2.4, glycine/NaCl). The low pH eluate was counted separately from the cell pellet, and percent internalization was calculated as previously described (McDevitt M R, et al., Cancer Res 2000; 60:6095-100).

### Preparation of Antibody-Drug Conjugates

The synthesis and design of the linkers and the conjugation of the linker to the cytotoxic drug were carried out as described in U.S. Pat. Nos. 6,884,889 and 6,214,345, each of which is herein incorporated by reference in its entirety. The conjugation of mAbs with maleimidocaproyl (mc)-valine (Val)-citrulline (Cit)-monomethyl auristatin E (MMAE) was performed as described (Doronina S O, et al., Nat. Biotechnology. 2003; 21:778-84). PSMA mAb and isotype-control human IgG1 (Calbiochem, San Diego, Calif.) in PBS containing 50 mM borate, pH 8.0, were treated with dithiothreitol (DTT) (10 mM final) at 37° C. for 30 min. The final reaction concentrations were 7.5 mL-8.0 ml, 1 mL 0.5 M sodium borate pH 8 and 0.5 M NaCl, 1 mL 100 mM DTT, and 0.5 mL or 0 ml, respectively, of PBS. This solution was incubated at 40° C. for 1 hr, and the antibody purified on a gel filtration column. The column was equilibrated with 10 mM DTPA in PBS at 10 mL/min, loaded with 10.0 mL of the antibody reduction mixture, and eluted at 8 mL/min in PBS/DTPA buffer. The concentration of antibody-cysteine thiols produced was determined by titrating with 5,5′-dithio-bis-(2-nitrobenzoic acid) (DTNB) (Pierce Chemical Co., Rockford, Ill.). An equivalent chemical can be obtained from Sigma (St. Louis, Mo.).

The fully reduced mAb Abgenix 4.40.2 (22.6 mL of 7.8 μM mAb, 75.6 μM cysteine thiol) was partially reoxidized with 35.43 μL of 10 mM DTNB, and the fully reduced mAb AB-PG1-XG1-006 (25.1 mL of 11.2 μM mAb, 95.8 μM cysteine thiol) was partially reoxidized with 56.27 μL of 10 mM DTNB. The color of the solution immediately turned yellow.

The drug mc-Val-Cit-paraaminobenzyl carbamate-MMAE (vcMMAE) was then conjugated to the partially reoxidized mAbs as follows: the mAbs were first cooled to 0° C. vcMMAE (5 molar equivalents per antibody: 89.7 and 140.6 μL, respectively, of a 10 mM stock solution of vcMMAE) was dissolved in 5 mL acetonitrile, then added to the antibody solution while carefully vortexing. The reaction mixtures were incubated on ice. No additional color change was observed. The reaction mixtures were quenched with 20 molar equivalents of cysteine/drug. The conjugate was purified using a gel-filtration column at 4° C. and eluted with PBS at 8.0 mL/min. The ADCs were determined to have ≥98% monomeric mAb containing 3.0-3.5 drugs per mAb using published methods (Doronina S O, et al., Nat Biotechnol. 2003; 21:778-84).

Alternatively, the conjugation of mAbs with maleimidocaproyl (mc)-valine (Val)-citrulline (Cit)-monomethyl auristatin E (MMAE) was performed as described (Doronina S O, et al., Nat. Biotechnology. 2003; 21:778-84). PSMA mAb and isotype-control human IgG1 (Calbiochem, San Diego, Calif.) in PBS containing 50 mM borate, pH 8.0, were treated with dithiothreitol (DTT) (10 mM final) at 37° C. for 30 min. The mAbs were exchanged into PBS containing 1 mM DTPA (Aldrich, Milwaukee, Wis.) by passage through a Sephadex G-25 column (Amersham Biosciences, Piscataway, N.J.). The mAb solutions were chilled to 4° C. and combined with the maleimido drug derivative in cold CH3CN. After 1 hour, the reactions were quenched with excess cysteine, and the conjugates were concentrated and exchanged into PBS buffer. The ADCs were determined to have ≥98% monomeric mAb containing 3.0-3.5 drugs per mAb using published methods (Doronina S O, et al., Nat Biotechnol. 2003; 21:778-84).

Reactivity of ADCs with Cell-Surface PSMA

Binding of PSMA mAb and ADC to 3T3™-PSMA and parental 3T3™ cells was analyzed using a FACSCalibur flow cytometer (BD Bioscience, San Diego, Calif.). Briefly, 2×10⁵ 3 T3™-PSMA (or 3T3™) cells were incubated with different concentrations of mAb or ADC on ice for 1 h. After washing, the presence of bound antibody was detected using goat anti-human IgG-FITC (Caltag Laboratories, Burlingame, Calif.). Isotype-control antibody and ADC were examined in parallel.

### In Vitro Cytotoxicity Assay

PSMA-positive cells (C4-2, LNCaP™ or 3T3™-PSMA) and PSMA-negative cells (PC-3™ or 3T3™) were added to 96-well microplates (Falcon, BD Biosciences, San Jose, Calif.) at 2.5×10³ cells/well and incubated overnight at 37° C. and 5% CO₂. Cells were then incubated with serially diluted ADCs for 4 days. The cell culture medium was replaced with fresh medium containing 10% Alamar Blue (Biosource International, Camarillo, Calif.), and cells were incubated for 4 h. Plates were then read on a fluorescence plate reader using an excitation wavelength of 530 nm and an emission wavelength of 590 nm. Cell survival was compared in treated and untreated cultures, and the concentration of ADC required for 50% cell kill (IC₅₀ value) was determined.

### Xenograft Model of Androgen-Independent Prostate Cancer

All animal studies were carried out in accordance with Animal Care and Use Committee guidelines. Athymic male nude mice (National Cancer Institute, Frederick, Md.) 6-8 weeks in age were implanted with an intramuscular injection of 5×10⁶ C4-2 cells mixed with 50% Matrigel (Beckon Dickinson Labware, Bedford, Mass.) into the left hind-leg as described (McDevitt M R, et al., Cancer Res 2000; 60:6095-100). Approximately 1 day prior to initiation of treatment, animals were randomized according to serum levels of prostate-specific antigen (PSA) as measured by ELISA (Medicorp, Montreal, Quebec, Canada). ADC, mAbs and vehicle control were administered via tail vein injection. In the first series of experiments, mice were treated in groups of 6 with 2 or 10 mg/kg PSMA ADC or with vehicle control. Treatment was initiated 17 days post-implantation and consisted of 3 injections at 4-day intervals (q4d×3). The second series of experiments examined dose levels of 0, 3 or 6 mg/kg. Treatment was initiated 14 days post-implantation and consisted of 6 injections at 4-day intervals (q4d×6). Animals were monitored for their physical appearance, body weight, PSA level and tumor size. Survival rates were recorded throughout the studies.

### Statistical Analyses

Treatment effects were examined for significance via t-tests (for PSA levels) or log-rank tests (for animal survival) using two-tailed, paired analyses. Data were considered significant when P<0.05.

### Results

Internalization of PSMA mAb into Human Prostate Cancer Cells

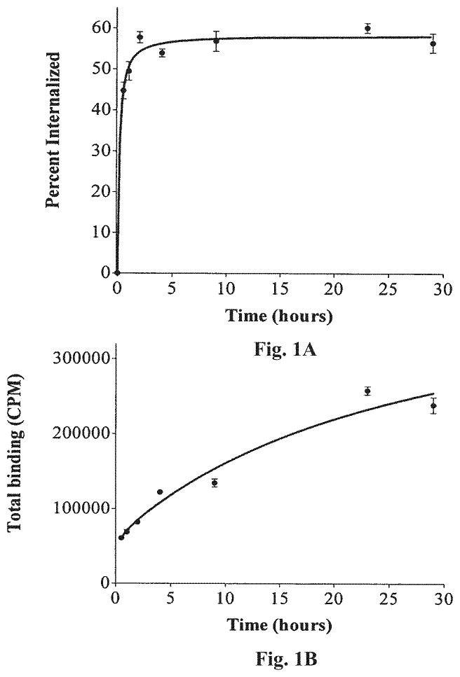

Internalization was examined using ¹¹¹In-labeled PSMA mAb and C4-2 cells. Total binding and percent internalization over time are illustrated in FIG. 1. Over half of the bound mAb was internalized within 2 h (FIG. 1A). Total binding increased over time, presumably due to PSMA recycling (FIG. 1B). Thus, the PSMA mAb is readily internalized into PSMA-expressing cells.

Reactivity of the PSMA ADC with PSMA-Expressing Cells

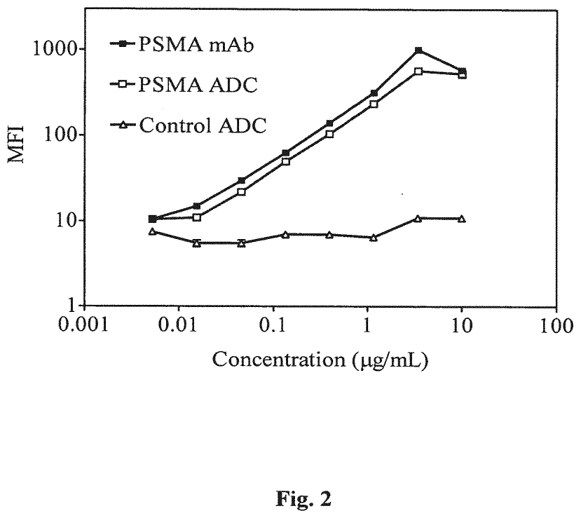

Flow cytometry was used to compare the binding of PSMA mAb and ADC. The unmodified mAb and ADC demonstrated comparable levels of binding to 3T3™-PSMA over a broad range of dilutions (FIG. 2). Neither the maximal amount of binding nor the concentration required for half-maximal binding was appreciably affected by conjugation. No significant binding was observed for the isotype-control ADC or antibody on 3T3™-PSMA cells or for PSMA mAb or ADC on parental 3T3™ cells.

### In Vitro Potency and Selectivity of the PSMA ADC

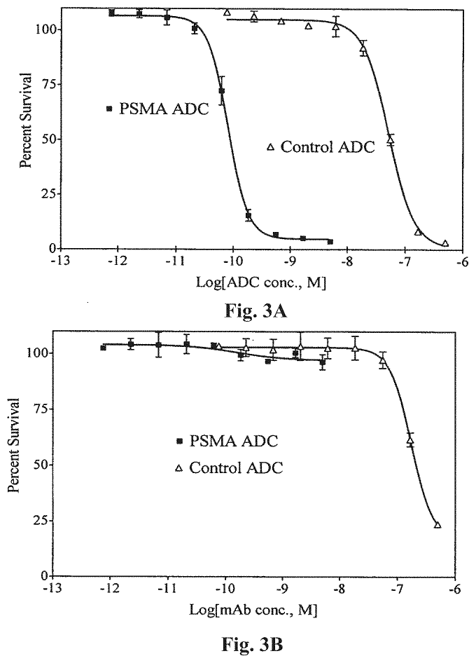

PSMA and control ADCs were tested for cytotoxicity in vitro against human prostate cancer cells lines and 3T3™-PSMA cells. FIG. 3 illustrates dose-response curves for PSMA-positive C4-2 cells and PSMA-negative PC-3™ cells in a representative experiment, and IC₅₀ values for the various cell lines are listed in Table 2. The PSMA ADC potently eliminated all PSMA-positive cell lines examined at IC₅₀ values of 65-210 pM, whereas these concentrations had no effect on PSMA-negative cells. In contrast, nearly 1000-fold higher concentrations were required for the control ADC, whose activity was independent of PSMA expression (FIG. 3 and Table 2).

TABLE 2
Summary of in vitro cytotoxicity (IC₅₀ values in pM)

| | | |
|---|---|---|
| C4-2 | LNCaP ™ | 3T3 ™-PSMA |

| | | | | | | |
|---|---|---|---|---|---|---|
| PSMA ADC | 65 ± 19 | (n = 3) | 83 ± 21 | (n = 2) | 208 ± 37 | (n = 3) |
| Control ADC | 54,954 | (n = 1) | 72,444 | (n = 1) | 154,880 | (n = 1) |

| | | | |
|---|---|---|---|
| Selectivity* | 848 | 877 | 744 |

*Selectivity equals the ratio of IC₅₀ values observed for the PSMA ADC and control ADC.

### Efficacy of the PSMA ADC in a Xenograft Model of Androgen-Independent Prostate Cancer

In vivo efficacy of the PSMA ADC was evaluated in a mouse model of androgen-independent human prostate cancer. Nude mice were engrafted with C4-2 cells intramuscularly in the left hind-leg. Approximately 14-17 days later, serum PSA levels were measured and used to randomly assign animals to treatment groups. Animals were treated intravenously with the PSMA ADC, and animals were monitored for tumor burden, PSA levels and other parameters for as long as 500 days.

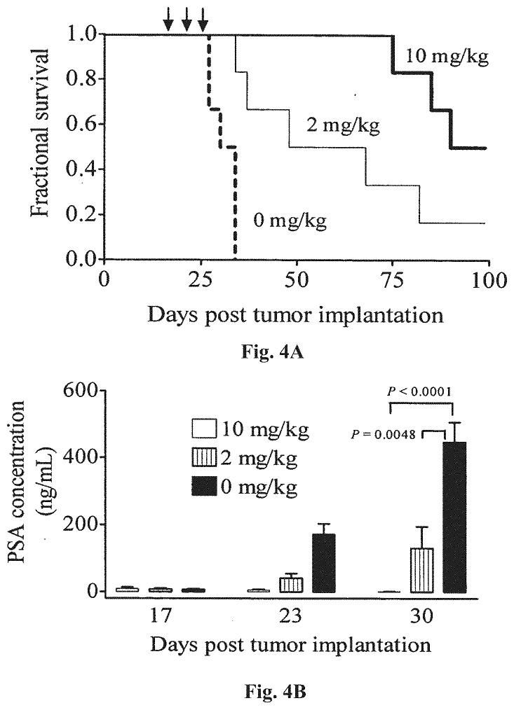

In the first experiment, animals were treated q4d×3 with 0, 2 or 10 mg/kg PSMA ADC. Left untreated, tumors grew rapidly and animals had a median survival of 32 days. In contrast, the groups treated with 2 mg/kg and 10 mg/kg PSMA ADC had median survivals of 58 days (P=0.0035) and 94.5 days (P=0.0012), respectively (Table 3, FIG. 4A). The PSMA ADC treatment significantly improved median survival up to 4.5-fold in a dose-dependent fashion. There was no evidence of treatment-related toxicity.

Serum PSA levels were measured over time by ELISA. FIG. 4B depicts the mean PSA concentration in each group at study days 17, 23 and 30. Treatment at 10 mg/kg reduced PSA levels >10-fold from 8.8±11.7 ng/mL at day 17 to 0.7±0.9 ng/mL at day 30, whereas PSA levels in the control group increased >60-fold over the same time period. An intermediate response was observed at 2 mg/kg PSMA ADC. The differences in PSA levels at day 30 were significant for both the 2 mg/kg (P=0.0048) and 10 mg/kg (P=0.0006) dose groups. Three of six animals in the 10 mg/kg group had undetectable PSA through day 52 of the study.

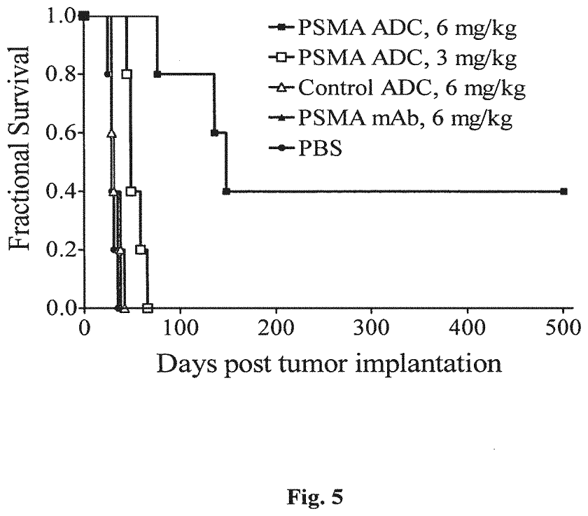

To extend these findings, a second PSMA ADC study was conducted that also included unmodified mAb and isotype-control ADC. After randomization at day 14 with a mean PSA level of 2.0±1.1 ng/mL in each group (n=5), animals were treated with a regimen of q4d×6. Kaplan-Meier survival curves for each group are depicted in FIG. 5. Animals treated with vehicle control, 6 mg/kg unmodified PSMA mAb and 6 mg/kg control ADC had similar median survival times of 29, 31 and 31 days, respectively; and these differences were not significant. However, median survival was extended to 49 days and 148 days for animals treated with 3 mg/kg and 6 mg/kg PSMA ADC, respectively (Table 3). Treatment of the PSMA ADC group with 6 mg/kg improved post-randomization survival 7.9-fold relative to the control ADC group (P=0.0018). At day 500, 2 of 5 animals had no evidence of tumor, no measurable PSA and were considered to be cured by treatment. As in the first study, treatment had a significant impact on PSA levels on day 29 (P=0.0068 for 6 mg/kg PSMA and vehicle groups). Moreover, in the 6 mg/kg PSMA ADC group, serum PSA decreased to undetectable levels post-treatment and remained undetectable through day 63 in 4 of 5 animals. There was no overt toxicity associated with ADC therapy. Physical appearance and activity were unaffected by treatment, and body weights of treated and vehicle-control animals were not significantly different at any time point.

TABLE 3
Summary of median survival times of C4-2 tumor-bearing
animals treated with PSMA ADC

| | | Dose | Median survival | |
|---|---|---|---|---|
| | Test article | (mg/kg) | (days) | P value* |
| Study #1 | Vehicle | NA | 32 | NA |
| | PSMA ADC | 2 | 58 | 0.0035 |
| | PSMA ADC | 10 | 95 | 0.0010 |
| Study #2 | Vehicle | NA | 29 | NA |
| | PSMA mAb | 6 | 31 | 0.1869 |
| | Control ADC | 6 | 31 | 0.2970 |
| | PSMA ADC | 3 | 49 | 0.0018 |
| | PSMA ADC | 6 | 148 | 0.0018 |

*Compared to the vehicle control group in a two-sided log-rank analysis.
NA= not applicable.

### Example 2: Evaluation of PSMA mAb Conjugated to Three Different Drug-Linkers

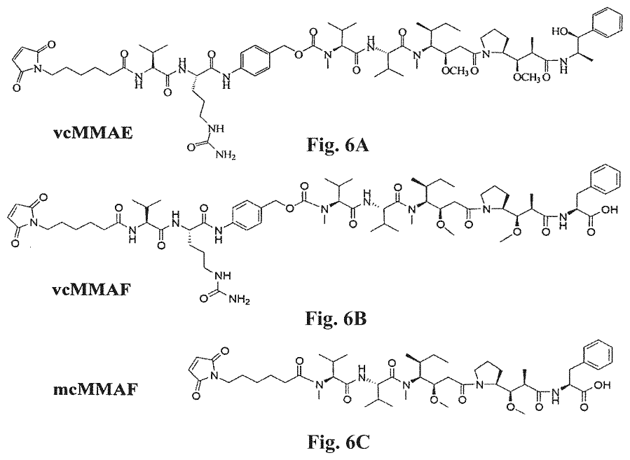

The PSMA mAb when conjugated to vcMMAE and two other drug-linkers, vcMMAF and mcMMAF, was evaluated. The full chemical structures of three different drug-linkers are illustrated in FIG. 6.

### Preparation of Three Drug-Linker Conjugates of PSMA mAb

The three drug-linkers were directly conjugated to PSMA mAb via a thioether bond to prepare approximately four drugs per antibody conjugates. Partial reduction of the mAb interchain disulfides proceeded with a slight excess of tris(2-carboxyethyl)phosphine (TCEP) at pH 7.2 and 37° C. and subsequent conjugation of the free thiols with drug-linkers was quantitative. Briefly, the PSMA mAb (10 mg, 67.5 nmol in PBS) was incubated at 37° C. with 1 mM DTPA and 169 nmol of TCEP for 90 min. At three time points during the incubation (30, 60 and 90 minutes), aliquots of 50 μg mAb were removed and reacted with an excess of vcMMAE. Analysis of the resulting ADCs by hydrophobic interaction chromatography allowed the progress of the reduction to be followed. The results indicated that the mAb was rapidly reduced under the above conditions, being essentially complete after 1 hour. Furthermore, the extent of reduction resulted in an average drug loading of 5 drugs/mAb.

To prepare a 4-loaded ADC with drug-linkers from the above partially reduced mAb, 0.5 equivalents of DTNB were added to re-oxidize the mAb population back to the desired level. Then, 3 mg of this material (20.3 nmol) was reacted with 101 nmol of vcMMAE, vcMMAF or mcMMAF in a 15% dimethyl sulfoxide (DMSO) reaction solution. This reaction proceeded for 1 hour at 0° C. and was then quenched with a 20-fold excess of N-acetyl cysteine. The ADCs were separated from unreacted drug and other small molecule impurities by size exclusion chromatography (SEC) on a PD-10 column (Amersham Biosciences/GE Healthcare, Piscataway, N.J.) and concentrated with a centrifugal concentration device (30 kD MWCO) (Amicon Bioseparations, Millipore Corporation, Bedford, Mass.).

A summary of the characterization of three drug-linker conjugates is provided in Tables 4-6 for vcMMAE, vcMMAF and mcMMAF, respectively. For each of the three drug-linkers, ADC contains approximately 4 drugs per mAb, as determined by H/L-chain loading distribution and species distribution, and <2% free drug as determined using reversed phase (RP) HPLC. For all conjugates, no aggregates were detected by SEC-HPLC. In addition, the overall mAb yields were 70-80%.

TABLE 4
Conjugate Certificate of Testing
699028A
PSMA mAb vcMMAE
Partial Reduction

| Assay | Method | Result |
|---|---|---|
| mAb Concentration | UV | 3.3 |
| mg/mL | | |
| Drug/mAb | H/L-Chain Loading | 4.3 |
| mol/mol | Distribution (PLRP) | |
| | Species Distribution | |
| | (HIC) | |
| Unconjugated Drug | RP-HPLC | <0.5 |
| % of total drug | | |
| Size Homogeneity | SEC-HPLC | Not detected |
| % Aggregate | | |
| Molar Ratio Distribution | HIC-HPLC | 3.5% 0 drugs/Ab |
| % of total | | 19.4% 2 drugs/Ab |
| | | 39.6% 4 drugs/Ab |
| | | 21.0% 6 drugs/Ab |
| | | 11.7% 8 drugs/Ab |
| Denatured Antibody | PLRP-HPLC | 31.3% L0 |
| | | 68.7% L1 |
| | | 10.7% H0 |
| | | 40.4% H1 |
| | | 25.1% H2 |
| | | 23.7% H3 |

TABLE 5
Conjugate Certificate of Testing
699028B
PSMA mAb vcMMAF
Partial Reduction

| Assay | Method | Result |
|---|---|---|
| mAb Concentration | UV | 3.1 |
| mg/mL | | |
| Drug/mAb | H/L-Chain Loading | 4.4 |
| mol/mol | Distribution (PLRP) | |
| | Species Distribution | |
| | (HIC) | |
| Unconjugated Drug | RP-HPLC | <0.5 |
| % of total drug | | |
| Size Homogeneity | SEC-HPLC | Not detected |
| % Aggregate | | |
| Molar Ratio Distribution | HIC-HPLC | 3.3% 0 drugs/Ab |
| % of total | | 18.5% 2 drugs/Ab |
| | | 39.0% 4 drugs/Ab |
| | | 22.2% 6 drugs/Ab |
| | | 13.5% 8 drugs/Ab |
| Denatured Antibody | PLRP-HPLC | 29.0% L0 |
| | | 71.0% L1 |
| | | 9.9% H0 |
| | | 40.2% H1 |
| | | 24.9% H2 |
| | | 25.0% H3 |

TABLE 6
Conjugate Certificate of Testing
699028C
PSMA mAb mcMMAF
Partial Reduction

| Assay | Method | Result |
|---|---|---|
| mAb Concentration | UV | 3.7 |
| mg/mL | | |
| Drug/mAb | H/L-Chain Loading | 4.4 |
| mol/mol | Distribution (PLRP) | |
| | Species Distribution | |
| | (HIC) | |
| Unconjugated Drug | RP-HPLC | 1.8 |
| % of total drug | | |
| Size Homogeneity | SEC-HPLC | Not detected |
| % Aggregate | | |
| Molar Ratio | HIC-HPLC | 3.8% 0 drugs/Ab |
| Distribution | | 22.4% 2 drugs/Ab |
| % of total | | 36.3% 4 drugs/Ab |
| | | 23.1% 6 drugs/Ab |
| | | 14.4% 8 drugs/Ab |
| Denatured Antibody | PLRP-HPLC | 32.7% L0 |
| | | 67.3% L1 |
| | | 14.7% H0 |
| | | 39.6% H1 |
| | | 23.8% H2 |
| | | 21.9% H3 |

### Potency and Selectivity of PSMA mAb Conjugates on Human Prostate Cancer Cells

In vitro cytotoxicity studies were conducted with PSMA-positive and PSMA-negative cell lines. Briefly, PSMA-positive cells (C4-2, LNCaP™ or 3T3™-PSMA) and PSMA-negative cells (PC-3™ or 3T3™) were added to 96-well microplates at 2.5×10³ cells/well and incubated overnight at 37° C. and 5% CO₂. Cells were then incubated with serially diluted ADCs for 4 days and assayed for percent cell kill compared to untreated controls using 10% Alamar Blue. The concentration of ADCs required for 50% cell kill (IC₅₀ value) was determined.

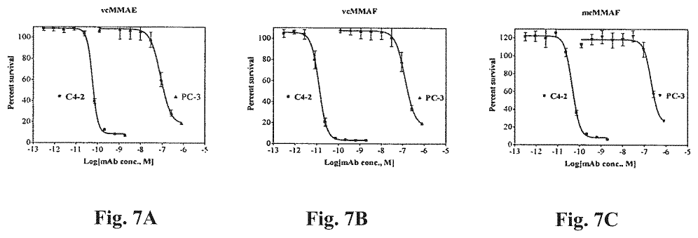

FIG. 7 illustrates dose-response curves of vcMMAE (FIG. 7A), vcMMAF (FIG. 7B) and mcMMAF (FIG. 7C) conjugates for PSMA-positive C4-2 cells and PSMA-negative PC-3™ cells in a representative experiment. A summary of the potency (IC₅₀) and selectivity on C4-2 and PC-3™ cell lines is listed in Table 7. The IC₅₀s on PSMA-expressing C4-2 cells were at picomolar concentrations of 11, 42, and 60 for vcMMAF, mcMMAF and vcMMAE conjugates, respectively. In contrast, the IC₅₀s on PC-3™ PSMA-negative cells were greater than 90 nM ranging from 94 to 264 nM. Based on the potency of each conjugate on PC-3™ and C4-2, the selectivity was calculated to be 13,636; 6,286 and 1,567 for vcMMAF, mcMMAF and vcMMAE conjugates, respectively. The vcMMAF conjugate was the most potent on the C4-2 PSMA positive cell line, and the mcMMAF was the least toxic over the PC-3™ control cell line. Compared to the vcMMAE conjugate, there was a 4-fold and 9-fold improvement in selectivity for mcMMAF and vcMMAF conjugates, respectively.

TABLE 7
Summary of in vitro potency (IC₅₀ values in pM) and selectivity

| | Potency (pM) | | Selectivity | Improvement |
|---|---|---|---|---|
| Drug-linker | C4-2 (n = 3) | PC-3 (n = 2) | (PC-3/C4-2) | over vcMMAE |
| vcMMAF | 11 | 150,000 | 13636 | 9-fold |
| mcMMAF | 42 | 264,000 | 6286 | 4-fold |
| vcMMAE | 60 | 94,000 | 1567 | — |

### Mechanism of Cell Killing by the PSMA mAb Drug Conjugate

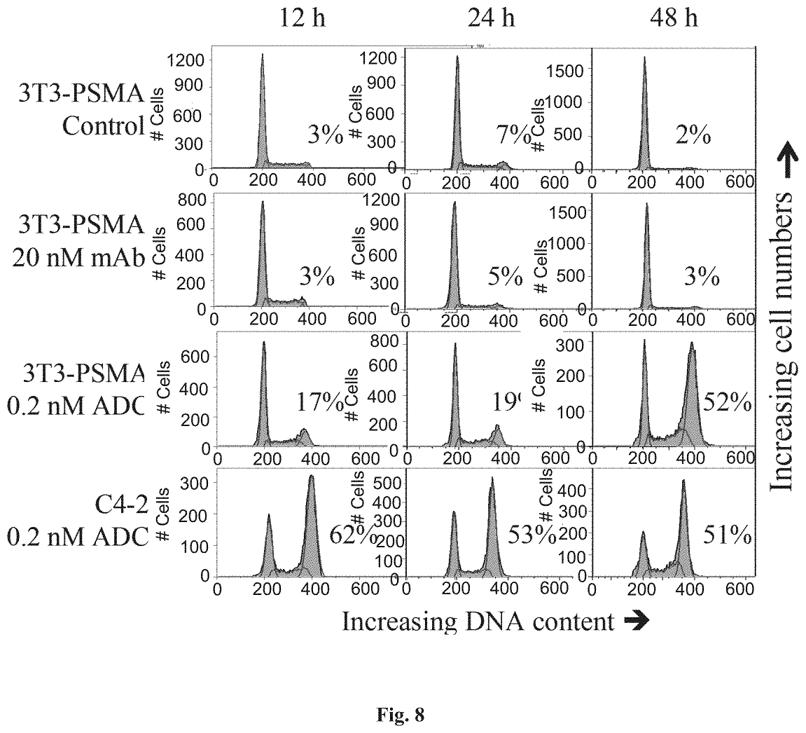

Cell-cycle analysis was performed to determine the mechanism of cytotoxicity mediated by MMAE-conjugated mAb. 3T3™-PSMA or C4-2 cells were cultured in the presence of 0.2 nM PSMA ADC or 20 nM unmodified PSMA mAb. Untreated cells served as a control culture. At 12 h, 24 h and 48 h, cells were stained with propidium iodide (PI) to detect total DNA and analyzed by flow cytometry. As indicated in FIG. 8, cells treated with PSMA ADC were arrested in G₂ phase. By 48 h post-treatment, the percent of cells with a duplicate set of chromosomes was >50% for the PSMA ADC cultures and 2% for untreated cultures. Cell-cycle arrest required the presence of the toxin, in this case MMAE, as only 3% of cells treated with unmodified mAb were in G₂/M phase at 48 h. The data demonstrate that treatment of prostate cancer cells with MMAE ADCs lead to G₂/M arrest and then apoptosis of target cells.

Each of the foregoing patents, patent applications and references that are recited in this application are herein incorporated in their entirety by reference. The recitation of the references is not intended to be an admission that any of the references is a prior art reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

1. An antibody-drug conjugate comprising:
an antibody or antigen-binding fragment thereof, which binds to prostate-specific membrane antigen (PSMA), conjugated to monomethylauristatin norephedrine or monomethylauristatin phenylalanine, wherein the antibody-drug conjugate has a PC-3™ cell to C4-2 or LNCaP™ cell selectivity of at least 250.

2. The antibody-drug conjugate of claim 1, wherein the antibody or antigen-binding fragment thereof is a monoclonal antibody or antigen-binding fragment thereof that specifically binds PSMA, optionally wherein the monoclonal antibody or antigen-binding fragment thereof binds an extracellular domain or conformational epitope of PSMA.

3-4. (canceled)

5. The antibody-drug conjugate of claim 1, wherein the antibody or antigen-binding fragment thereof
(i) competitively inhibits the specific binding of a second antibody to its target epitope on PSMA, optionally wherein the second antibody is selected from the group consisting of:
(a) PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1, Abgenix 4.152.1, and antibodies comprising: a heavy chain encoded by a nucleic acid molecule comprising a coding region or regions of a nucleotide sequence selected from the group consisting of nucleotide sequences set forth as SEQ ID NOs: 2-7, and a light chain encoded by a nucleic acid molecule comprising a coding region or regions of a nucleotide sequence selected from the group consisting of nucleotide sequences set forth as SEQ ID NOs: 8-13,
(b) AB-PG1-XG1-006, AB-PG1-XG1-026, and antibodies comprising: a heavy chain encoded by a nucleic acid molecule comprising a coding region or regions of a nucleotide sequence selected from the group consisting of nucleotide sequences set forth as SEQ ID NOs: 2 and 3, and a light chain encoded by a nucleic acid molecule comprising a coding region or regions of a nucleotide sequence selected from the group consisting of nucleotide sequences set forth as SEQ ID NOs: 8 and 9, or
(ii) binds to an epitope on PSMA defined by an antibody selected from the group consisting of PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1, Abgenix 4.152.1 and antibodies comprising:
(a) a heavy chain encoded by a nucleic acid molecule comprising a coding region or regions of a nucleotide sequence selected from the group consisting of nucleotide sequences set forth as SEQ ID NOs: 2-7, and
(b) a light chain encoded by a nucleic acid molecule comprising a coding region or regions of a nucleotide sequence selected from the group consisting of nucleotide sequences set forth as SEQ ID NOs: 8-13.

6-9. (canceled)

10. The antibody-drug conjugate of claim 1, wherein the antibody is encoded by a nucleic acid molecule comprising a nucleotide sequence that is at least 90%, at least 95%, at least 97%, at least 98%, or at least 99% identical to a nucleotide sequence encoding an antibody selected from the group consisting of: AB-PG1-XG1-006, AB-PG1-XG1-026 and antibodies comprising:
(a) a heavy chain encoded by a nucleic acid molecule comprising a coding region or regions of a nucleotide sequence selected from the group consisting of nucleotide sequences set forth as SEQ ID NOs: 2 and 3, and
(b) a light chain encoded by a nucleic acid molecule comprising a coding region or regions of a nucleotide sequence selected from the group consisting of nucleotide sequences set forth as SEQ ID NOs: 8 and 9,
optionally wherein the antibody or antigen-binding fragment thereof is AB-PG1-XG1-006, AB-PG1-XG1-026, or an antigen-binding fragment thereof.

11-15. (canceled)

16. The antibody-drug conjugate of claim 1, wherein the antibody or antigen-binding fragment thereof is selected from the group consisting of antibodies comprising:
(a) a heavy chain encoded by a nucleic acid molecule comprising a coding region or regions of a nucleotide sequence selected from the group consisting of nucleotide sequences set forth as SEQ ID NOs: 2 and 3, and
(b) a light chain encoded by a nucleic acid molecule comprising a coding region or regions of a nucleotide sequence selected from the group consisting of nucleotide sequences set forth as SEQ ID NOs: 8 and 9, and
antigen-binding fragments thereof, optionally wherein the antibody or antigen-binding fragment thereof comprises:
(i) a heavy chain encoded by a nucleic acid molecule comprising a coding region or regions of a nucleotide sequence set forth as SEQ ID NO: 2, and a light chain encoded by a nucleic acid molecule comprising a coding region or regions of a nucleotide sequence set forth as SEQ ID NO: 8, and antigen binding fragments thereof; or
(ii) a heavy chain encoded by a nucleic acid molecule comprising a coding region or regions of a nucleotide sequence set forth as SEQ ID NO: 3, and a light chain encoded by a nucleic acid molecule comprising a coding region or regions of a nucleotide sequence set forth as SEQ ID NO: 9, and antigen binding fragments thereof.

17-18. (canceled)

19. The antibody-drug conjugate of claim 1, wherein the antibody or antigen-binding fragment thereof is IgG1, IgG2, IgG3, IgG4, IgM, IgA1, IgA2, IgAsec, IgD, IgE or has immunoglobulin constant and/or variable domain of IgG1, IgG2, IgG3, IgG4, IgM, IgA1, IgA2, IgAsec, IgD or IgE.

20. The antibody-drug conjugate of claim 1, wherein the antibody is a monoclonal antibody, a humanized antibody, a human antibody, a recombinant antibody, a chimeric antibody, a bispecific antibody, and/or a multispecific antibody.

21-25. (canceled)

26. The antibody-drug conjugate of claim 1, wherein the antigen-binding fragment is a Fab fragment, a F(ab′)₂ fragment, a CDR3-containing fragment, or a Fv fragment.

27. (canceled)

28. The antibody-drug conjugate of claim 1, wherein the PC-3™ cell to C4-2 or LNCaP™ cell selectivity is at least 500, 1000, 2500, 6000 or 13,000.

29. The antibody-drug conjugate of claim 1, wherein the antibody or antigen-binding fragment thereof is conjugated to:
(i) at least 3 monomethylauristatin norephedrine or monomethylauristatin phenylalanine molecules; or
(ii) at least 4 monomethylauristatin norephedrine or monomethylauristatin phenylalanine molecules.

30. (canceled)

31. The antibody-drug conjugate of claim 1, wherein the monomethylauristatin norephedrine or monomethylauristatin phenylalanine is conjugated to the antibody or antigen-binding fragment thereof with a compound of the formula:
-Aₙ-Yₘ—Zₘ—Xₙ—Wₙ—
wherein, A is a carboxylic acyl unit;
Y is an amino acid;
Z is an amino acid;
X and W are each a self-immolative spacer;
n is an integer of 0 or 1; and
m is an integer of 0 or 1, 2, 3, 4, 5 or 6, optionally wherein:
(i) A is 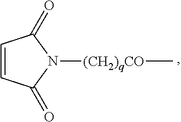
in which q is 1-10;
(ii) A is 4-(N-succinimidomethyl)cyclohexane-1-carbonyl, m-succinimidobenzoyl, 4-(p-succinimidophenyl)-butyryl, 4-(2-acetamido)benzoyl, 3-thiopropionyl, 4-(1-thioethyl)-benzoyl, 6-(3-thiopropionylamido)-hexanoyl or maleimide caproyl;
(iii) A is maleimide caproyl;
(iv) Y is alanine, valine, leucine, isoleucine, methionine, phenylalanine, tryptophan or proline;
(v) Y is valine;
(vi) Z is lysine, lysine protected with acetyl or formyl, arginine, arginine protected with tosyl or nitro groups, histidine, ornithine, ornithine protected with acetyl or formyl, or citrulline;
(vii) Z is citrulline;
(viii) Yₘ—Zₘ is valine-citrulline;
(ix) Yₘ—Zₘ is a protein sequence which is selectively cleavable by a protease;
(x) X is a compound having the formula: 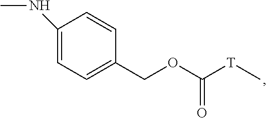
in which T is O, N, or S;
(xi) X is a compound having the formula: —HN—R¹—COT, in which R¹ is C₁-C₅ alkyl, T is O, N or S;
(xii) X is a compound having the formula: 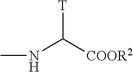
in which T is O, N, or S, R² is H or C₁-C₅ alkyl;
(xiii) X is p-aminobenzylcarbamoyloxy;
(xiv) X is p-aminobenzylalcohol;
(xv) X is p-aminobenzylcarbamate;
(xvi) X is p-aminobenzyloxycarbonyl.
(xvii) X is γ-aminobutyric acid; α,α-dimethyl γ-aminobutyric acid or β,β-dimethyl γ-aminobutyric acid;
(xviii) W is 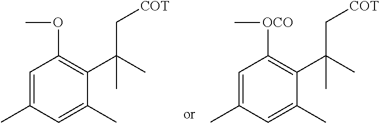
in which T is O, S or N; and/or
(xix) m and n are 0.

32-50. (canceled)

51. The antibody-drug conjugate of claim 31, which is:
(i) AB-PG1-XG1-006-maleimide caproyl-valine-citrulline-p-aminobenzyloxycarbonyl-monomethylauristatin norephedrine
(ii) AB-PG1-XG1-006-maleimide caproyl-valine-citrulline-p-aminobenzyloxycarbonyl-monomethylauristatin phenylalanine;
(iii) AB-PG1-XG1-006-maleimide caproyl-monomethylauristatin phenylalanine;
(iv) AB-PG1-XG1-026-maleimide caproyl-valine-citrulline-p-aminobenzyloxycarbonyl-monomethylauristatin norephedrine;
(v) AB-PG1-XG1-026-maleimide caproyl-valine-citrulline-p-aminobenzyloxycarbonyl-monomethylauristatin phenylalanine; or
(vi) AB-PG1-XG1-026-maleimide caproyl-monomethylauristatin phenylalanine.

52-56. (canceled)

57. The antibody-drug conjugate of claim 1, wherein the antibody-drug conjugate:
(i) binds live cells or endothelial cells of the neovasculature of a tumor; optionally wherein the cells are tumor cells, optionally wherein the tumor is a prostate tumor;
(ii) does not require cell lysis to bind PSMA;
(iii) leads to cell-cycle arrest;
(iv) inhibits the growth of PSMA-expressing cells; and/or
(v) mediates specific cell killing of PSMA-expressing cells with an IC₅₀ of:
(a) less than 1×10⁻¹⁰M;
(b) less than 1×10⁻¹¹M;
(c) less than 1×10⁻¹²M;
(d) 11 to 208×10⁻¹²M;
(e) 42 to 208×10⁻¹²M;
(f) 60 to 208×10⁻¹²M;
(g) 65 to 208×10⁻¹²M;
(h) 11×10⁻¹²M;
(i) 42×10⁻¹²M;
(j) 60×10⁻¹²M; and/or
(k) 83×10⁻¹²M.

58-73. (canceled)

74. The antibody-drug conjugate of claim 1, wherein the antibody-drug conjugate when administered to mice with a regimen of q4d×6 at a dose of 6 mg/kg effects a cure rate of at least 20%, 30%, 40% or 50%.

75. The antibody-drug conjugate of claim 1,
(i) bound to a label, optionally wherein the label is a fluorescent label, an enzyme label, a radioactive label, a nuclear magnetic resonance active label, a luminescent label or a chromophore label; and/or
(ii) packaged in lyophilized form, packaged in aqueous medium, or in a sterile form.

76-79. (canceled)

80. A composition comprising:
(i) the antibody-drug conjugate of claim 1 and a pharmaceutically acceptable carrier, excipient or stabilizer;
(ii) a combination of two or more different antibody-drug conjugates according to claim 1 and a pharmaceutically acceptable carrier, excipient or stabilizer; or
(iii) one or more antibody-drug conjugates of claim 1 and one or more unconjugated anti-PSMA antibodies;
optionally wherein the composition according to (i) or (ii) further comprises an antitumor agent, an immunostimulatory agent, an immunomodulator, a corticosteroid, or a combination thereof, optionally wherein:
(a) the antitumor agent is a cytotoxic agent, an agent that acts on tumor neovasculature or a combination thereof;
(b) the antitumor agent is docetaxel;
(c) the immunomodulator is a cytokine, chemokine, adjuvant or a combination thereof;
(d) the immunostimulatory agent is interleukin-2, α-interferon, γ-interferon, tumor necrosis factor-α, immunostimulatory oligonucleotides or a combination thereof; and/or
(e) the corticosteroid is prednisone or hydrocortisone.

81-88. (canceled)

89. A method for inhibiting the growth of a PSMA-expressing cell comprising:
contacting the PSMA-expressing cell with an amount of the antibody-drug conjugate of claim 1 effective to inhibit the growth of the PSMA-expressing cell optionally wherein the PSMA-expressing cell is a prostate tumor cell or a cell of the neovasculature of a non-prostate tumor, and/or an androgen-dependent cell or an androgen-independent cell, optionally wherein the method further comprises contacting the PSMA-expressing cell with an antitumor agent, an immunostimulatory agent, an immunomodulator, corticosteroid or a combination thereof, optionally wherein:
(i) the antitumor agent is a cytotoxic agent, an agent that acts on tumor neovasculature, or a combination thereof;
(ii) the antitumor agent is docetaxel;
(iii) the immunomodulator is a cytokine, chemokine, adjuvant or a combination thereof;
(iv) the immunostimulatory agent is interleukin-2, α-interferon, γ-interferon, tumor necrosis factor-α, immunostimulatory oligonucleotides or a combination thereof; and/or
(v) the corticosteroid is prednisone or hydrocortisone.

90-98. (canceled)

99. A method for effecting cell-cycle arrest in a PSMA-expressing cell comprising:
contacting the PSMA-expressing cell with an amount of the antibody-drug conjugate of claim 1 effective to lead to cell-cycle arrest in the PSMA-expressing cell.

100. A method for treating a PSMA-mediated disease comprising:
administering to a subject having a PSMA-mediated disease an amount of the antibody-drug conjugate of claim 1 effective to treat the PSMA-mediated disease, optionally wherein:
(i) the PSMA-mediated disease is cancer, optionally wherein the cancer is prostate or non-prostate cancer, optionally wherein the non-prostate cancer is bladder cancer, pancreatic cancer, lung cancer, kidney cancer, sarcoma, breast cancer, brain cancer, neuroendocrine carcinoma, colon cancer, testicular cancer or melanoma; and/or
(ii) wherein the therapeutic agent is a vaccine, optionally wherein the vaccine immunizes the subject against PSMA,
optionally wherein the method further comprises co-administering one or more other therapeutic agents to treat the PSMA-mediated disease, wherein the co-administration is before, during or after the administration of the antibody-drug conjugate, optionally wherein the one or more other therapeutic agents is an antitumor agent, an immunostimulatory agent, an immunomodulator, corticosteroid, or a combination thereof, optionally wherein:
(i) the antitumor agent is a cytotoxic agent, an agent that acts on tumor neovasculature or a combination thereof;
(ii) the antitumor agent is docetaxel;
(iii) the immunomodulator is a cytokine, chemokine, adjuvant or a combination thereof;
(iv) the immunostimulatory agent is interleukin-2, α-interferon, γ-interferon, tumor necrosis factor-α, immunostimulatory oligonucleotides or a combination thereof; and/or
(v) the corticosteroid is prednisone or hydrocortisone.

101-115. (canceled)

116. A method for inhibiting the growth of a tumor comprising:
contacting the PSMA-expressing cells of the neovasculature of the tumor with an amount of the antibody-drug conjugate of claim 1 effective to inhibit the growth of the tumor, optionally wherein the method further comprises contacting the PSMA-expressing cells with an antitumor agent, an immunostimulatory agent, an immunomodulator, corticosteroid or a combination thereof, optionally wherein:
(i) the antitumor agent is a cytotoxic agent, an agent that acts on tumor neovasculature or a combination thereof;
(ii) the immunomodulator is a cytokine, chemokine, adjuvant or a combination thereof; and/or
(iii) the immunostimulatory agent is interleukin-2, α-interferon, γ-interferon, tumor necrosis factor-α, immunostimulatory oligonucleotides or a combination thereof.

117-120. (canceled)


SEQUENCE LISTING (33 sequences)

SEQ ID NO: 1 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 2 - DNA, 7570 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggagttgggactgcgctggggcttcctcgttgctcttttaagaggtgtccagtgtcaggtgcaattggtggagtctgggggaggcgtggtccagcctgggaggtccctgagactctcctgtgcagcgtctggattcgccttcagtagatatggcatgcactgggtccgccaggctccaggcaaggggctggagtgggtggcagttatatggtatgatggaagtaataaatactatgcagactccgtgaagggccgattcaccatctccagagacaattccaagaacacgcagtatctgcaaatgaacagcctgagagccgaggacacggctgtgtattactgtgcgagaggcggtgacttcctctactactactattacggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 7597 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggggtcaaccgccatcctcaccatggagttggggctgcgctgggttctcctcgttgctcttttaagaggtgtccagtgtcaggtgcagctggtggagtctgggggaggcgtggtccagcctgggaggtccctgagactctcctgtgcagcgtctggattcaccttcagtaactatgtcatgcactgggtccgccaggctccaggcaaggggctggagtgggtggcaattatatggtatgatggaagtaataaatactatgcagactccgtgaagggccgattcaccatctccagagacaattccaagaacacgctgtatctgcaaatgaacagcctgagagccgaggacacggctgtgtattactgtgcgggtggatataactggaactacgagtaccactactacggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 7579 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggagttgggacttagctgggttttcctcgttgctcttttaagaggtgtccagtgtcaggtccagctggtggagtctgggggaggcgtggtccagcctgggaggtccctgagactctcctgtgcagcgtctggattcaccttcagtagctatggcatgcactgggtccgccaggctccaggcaaggggctggactgggtggcaattatttggcatgatggaagtaataaatactatgcagactccgtgaagggccgattcaccatctccagagacaattccaagaagacgctgtacctgcaaatgaacagtttgagagccgaggacacggctgtgtattactgtgcgagagcttgggcctatgactacggtgactatgaatactacttcggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 7558 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatcccaccatggggtcaaccgtcatcctcgccctcctcctggctgttctccaaggagtctgtgccgaggtgcagctggtgcagtctggagcagaggtgaaaaagcccggggagtctctgaagatctcctgtaagggttctggatacagctttaccagttactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacggatggcagcagctggcccctttgactactggggccagggaaccctggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 7576 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggagtttgggctgtgctggattttcctcgttgctcttttaagaggtgtccagtgtcaggtgcagctggtggagtctgggggaggcgtggtccagcctgggaggtccctgagactctcctgtgcagcctctggattcaccttcattagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgagagtattagtgggagctttatattattataactactacgggatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - DNA, 7561 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccggatctcaccatggagttggggctgagctgggttttcctcgttgctcttttaagaggtgtccagtgtcaggagcagctggtggagtctgggggaggcgtggtccagcctgggaggtccctgagactctcctgtgcagcgtctggattcaccttcagtacctatggcatgcactgggtccgccaggctccaggcaaggggctggagtgggtggcagttacatggcatgatggaagtaataaatactatgcagactccgtgaagggccgattcaccatctccagagacaactccaagaacacgctgtatctgcaaatgaacagcctgagagccgaggacacggctgtgtattactgtgcgagaggaggagtgggagcaacttactactactactacggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - DNA, 6082 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccctgctcagctcctgggactcctgctgctctggctcccagataccagatgtgacatccagatgacccagtctccatcctccctgtctgcatctgtaggagacagagtcaccatcacttgccgggcgagtcagggcattagcaattatttagcctggtatcagcagaaaacagggaaagttcctaagttcctgatctatgaagcatccactttgcaatcaggggtcccatctcggttcagtggcggtggatctgggacagatttcactctcaccatcagcagcctgcagcctgaagatgttgcaacttattactgtcaaaattataacagtgccccattcactttcggccctgggaccaaagtggatatcaaacgaactgtggctgcaccctctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 6082 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccccgctcagctcctggggctcctgctgctctgtttcccaggtgccagatgtgacatccagatgacccagtctccatcctcactgtctgcatctgtaggagacagagtcaccatcacttgtcgggcgagtcagggcattaccaattatttagcctggtttcagcagaaaccagggaaagcccctaagtcccttatctatgctgcatccagtttgcaaagtggggtcccatcaaagttcagcggcagtggatctgggacagatttcagtctcaccatcagcagcctgcagcctgaagattttgcaacttattactgccaacagtataatagttacccgatcaccttcggccaagggacacgactggagattaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - DNA, 6082 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccctgctcagctcctggggctcctgctgctctgtttcccaggtgccagatgtgacatccagatgacccagtctccatcctcactgtctgcatctgtaggagacagagtcaccatcacttgtcgggcgagtcagggcattagccattatttagcctggtttcagcagaaaccagggaaagcccctaagtccctgatctatgctgcatccagtttgcaaagtggggtcccatcaaagttcagcggcagtggatctgggacagatttcactctcaccatcagcagcctacagcctgaagattttgcaacttattactgccaacagtataatagtttcccgctcactttcggcggagggaccaaggtggagatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 11 - DNA, 6085 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccccgctcagcttctcttccttctgctactctggctcccagataccactggaggaatagtgatgacgcagtctccagccaccctgtctgtgtctccaggggaaagagccaccctctcctgcaggaccagtcagagtattggctggaacttagcctggtaccaacagaaacctggccaggctcccaggctcctcatctatggtgcatcttccaggaccactggtatcccagccaggttcagtggcagtgggtctgggacagagttcactctcaccatcagcagcctgcagtctgaagattctgcagtttattactgtcagcattatgataactggcccatgtgcagttttggccaggggaccgagctggagatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 12 - DNA, 6097 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccctgctcagctcctggggctgctaatgctctggatacctggatccagtgcagatattgtgatgacccagactccactctctctgtccgtcacccctggacagccggcctccatctcctgcaagtctagtcagagcctcctgcatagtgatggaaagacctttttgtattggtatctgcagaagccaggccagcctccacagctcctgatctatgaggtttccaaccggttctctggagtgccagataggttcagtggcagcgggtcagggacagatttcacactgaaaatcagccgggtggaggctgaggatgttgggctttattactgcatgcaaagtatacagcttccgctcactttcggcggagggaccaaggtggagatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 6094 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatggtgttgcagacccaggtcttcatttctctgttactctggatctctggtgcctacggggacatcgtgatgacccagtctccagactccctggctgtgtctctgggcgagagggccaccatcaactgcaagtccaaccagagtgtcttacacagctccaacaataagaactatttagcttggtaccagcagaaaccaggacagcctcctaaattgctcatttattgggcattcctccgggaatccggggtccctgaccgcttcagtggcagcgggtctgggacagatttcactctcaccatcagcagcctgcaggctgaagatgtggcagtttattactgtcaccaatattattctactttatatactttcggcggagggaccaaggtagagatcaaacgaacygtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - DNA, 481 bp, Artificial Sequence
  Includes BamHI/Bg1II cloning junction, signal peptide, V region, portion of C region and 3XbaI/NheI (heavy) or NheI (light) cloning junction
ggatctcaccatggagttgggactgcgctggggcttcctcgttgctcttttaagaggtgtccagtgtcaggtgcaattggtggagtctgggggaggcgtggtccagcctgggaggtccctgagactctcctgtgcagcgtctggattcgccttcagtagatatggcatgcactgggtccgccaggctccaggcaaggggctggagtgggtggcagttatatggtatgatggaagtaataaatactatgcagactccgtgaagggccgattcaccatctccagagacaattccaagaacacgcagtatctgcaaatgaacagcctgagagccgaggacacggctgtgtattactgtgcgagaggcggtgacttcctctactactactattacggtatggacgtctggggccaagggaccacggtcaccgtctcctcagcctccaccaagggcccatcggtcttccccctggcaccctctagc

SEQ ID NO: 15 - protein, 142 aa, Homo sapiens
MELGLRWGFLVALLRGVQCQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSS

SEQ ID NO: 16 - DNA, 463 bp, Artificial Sequence
  Includes BamHI/Bg1II cloning junction, signal peptide, V region, portion of C region and 3XbaI/NheI (heavy) or NheI (light) cloning junction
ggatctcaccatgagggtccctgctcagctcctgggactcctgctgctctggctcccagataccagatgtgacatccagatgacccagtctccatcctccctgtctgcatctgtaggagacagagtcaccatcacttgccgggcgagtcagggcattagcaattatttagcctggtatcagcagaaaacagggaaagttcctaagttcctgatctatgaagcatccactttgcaatcaggggtcccatctcggttcagtggcggtggatctgggacagatttcactctcaccatcagcagcctgcagcctgaagatgttgcaacttattactgtcaaaattataacagtgccccattcactttcggccctgggaccaaagtggatatcaaacgaactgtggctgcaccctctgtcttcatcttcccgccatctgatgagcagttgaaatctggaactgctagc

SEQ ID NO: 17 - protein, 127 aa, Homo sapiens
MRVPAQLLGLLLLWLPDTRCDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIK

SEQ ID NO: 18 - DNA, 508 bp, Artificial Sequence
  Includes BamHI/Bg1II cloning junction, signal peptide, V region, portion of C region and 3XbaI/NheI (heavy) or NheI (light) cloning junction
ggatctcaccatggggtcaaccgccatcctcaccatggagttggggctgcgctgggttctcctcgttgctcttttaagaggtgtccagtgtcaggtgcagctggtggagtctgggggaggcgtggtccagcctgggaggtccctgagactctcctgtgcagcgtctggattcaccttcagtaactatgtcatgcactgggtccgccaggctccaggcaaggggctggagtgggtggcaattatatggtatgatggaagtaataaatactatgcagactccgtgaagggccgattcaccatctccagagacaattccaagaacacgctgtatctgcaaatgaacagcctgagagccgaggacacggctgtgtattactgtgcgggtggatataactggaactacgagtaccactactacggtatggacgtctggggccaagggaccacggtcaccgtctcctcagcctccaccaagggcccatcggtcttccccctggcaccctctagc

SEQ ID NO: 19 - protein, 143 aa, Homo sapiens
MELGLRWVLLVALLRGVQCQVQLVESGGGVVQPGRSLRLSCAASGFTFSNYVMHWVRQAPGKGLEWVAIIWYDGSNKYYADSVKGRFTISRDNSKNTLYLQMNSLRAEDTAVYYCAGGYNWNYEYHYYGMDVWGQGTTVTVSS

SEQ ID NO: 20 - DNA, 463 bp, Artificial Sequence
  Includes BamHI/Bg1II cloning junction, signal peptide, V region, portion of C region and 3XbaI/NheI (heavy) or NheI (light) cloning junction
ggatctcaccatgagggtccccgctcagctcctggggctcctgctgctctgtttcccaggtgccagatgtgacatccagatgacccagtctccatcctcactgtctgcatctgtaggagacagagtcaccatcacttgtcgggcgagtcagggcattaccaattatttagcctggtttcagcagaaaccagggaaagcccctaagtcccttatctatgctgcatccagtttgcaaagtggggtcccatcaaagttcagcggcagtggatctgggacagatttcagtctcaccatcagcagcctgcagcctgaagattttgcaacttattactgccaacagtataatagttacccgatcaccttcggccaagggacacgactggagattaaacgaactgtggctgcaccatctgtcttcatcttcccgccatctgatgagcagttgaaatctggaactgctagc

SEQ ID NO: 21 - protein, 127 aa, Homo sapiens
MRVPAQLLGLLLLCFPGARCDIQMTQSPSSLSASVGDRVTITCRASQGITNYLAWFQQKPGKAPKSLIYAASSLQSGVPSKFSGSGSGTDFSLTISSLQPEDFATYYCQQYNSYPITFGQGTRLEIK

SEQ ID NO: 22 - DNA, 490 bp, Artificial Sequence
  Includes BamHI/Bg1II cloning junction, signal peptide, V region, portion of C region and 3XbaI/NheI (heavy) or NheI (light) cloning junction
ggatctcaccatggagttgggacttagctgggttttcctcgttgctcttttaagaggtgtccagtgtcaggtccagctggtggagtctgggggaggcgtggtccagcctgggaggtccctgagactctcctgtgcagcgtctggattcaccttcagtagctatggcatgcactgggtccgccaggctccaggcaaggggctggactgggtggcaattatttggcatgatggaagtaataaatactatgcagactccgtgaagggccgattcaccatctccagagacaattccaagaagacgctgtacctgcaaatgaacagtttgagagccgaggacacggctgtgtattactgtgcgagagcttgggcctatgactacggtgactatgaatactacttcggtatggacgtctggggccaagggaccacggtcaccgtctcctcagcctccaccaagggcccatcggtcttccccctggcaccctctagc

SEQ ID NO: 23 - protein, 145 aa, Homo sapiens
MELGLSWVFLVALLRGVQCQVQLVESGGGVVQPGRSLRLSCAASGFTFSSYGMHWVRQAPGKGLDWVAIIWHDGSNKYYADSVKGRFTISRDNSKKTLYLQMNSLRAEDTAVYYCARAWAYDYGDYEYYFGMDVWGQGTTVTVSS

SEQ ID NO: 24 - DNA, 463 bp, Artificial Sequence
  Includes BamHI/Bg1II cloning junction, signal peptide, V region, portion of C region and 3XbaI/NheI (heavy) or NheI (light) cloning junction
ggatctcaccatgagggtccctgctcagctcctggggctcctgctgctctgtttcccaggtgccagatgtgacatccagatgacccagtctccatcctcactgtctgcatctgtaggagacagagtcaccatcacttgtcgggcgagtcagggcattagccattatttagcctggtttcagcagaaaccagggaaagcccctaagtccctgatctatgctgcatccagtttgcaaagtggggtcccatcaaagttcagcggcagtggatctgggacagatttcactctcaccatcagcagcctacagcctgaagattttgcaacttattactgccaacagtataatagtttcccgctcactttcggcggagggaccaaggtggagatcaaacgaactgtggctgcaccatctgtcttcatcttcccgccatctgatgagcagttgaaatctggaactgctagc

SEQ ID NO: 25 - protein, 127 aa, Homo sapiens
MRVPAQLLGLLLLCFPGARCDIQMTQSPSSLSASVGDRVTITCRASQGISHYLAWFQQKPGKAPKSLIYAASSLQSGVPSKFSGSGSGTDFTLTISSLQPEDFATYYCQQYNSFPLTFGGGTKVEIK

SEQ ID NO: 26 - DNA, 469 bp, Artificial Sequence
  Includes BamHI/Bg1II cloning junction, signal peptide, V region, portion of C region and 3XbaI/NheI (heavy) or NheI (light) cloning junction
ggatcccaccatggggtcaaccgtcatcctcgccctcctcctggctgttctccaaggagtctgtgccgaggtgcagctggtgcagtctggagcagaggtgaaaaagcccggggagtctctgaagatctcctgtaagggttctggatacagctttaccagttactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacggatggcagcagctggcccctttgactactggggccagggaaccctggtcaccgtctcctcagcctccaccaagggcccatcggtcttccccctggcaccctctagc

SEQ ID NO: 27 - protein, 138 aa, Homo sapiens
MGSTVILALLLAVLQGVCAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARRMAAAGPFDYWGQGTLVTVSS

SEQ ID NO: 28 - DNA, 466 bp, Artificial Sequence
  Includes BamHI/Bg1II cloning junction, signal peptide, V region, portion of C region and 3XbaI/NheI (heavy) or NheI (light) cloning junction
ggatctcaccatgagggtccccgctcagcttctcttccttctgctactctggctcccagataccactggaggaatagtgatgacgcagtctccagccaccctgtctgtgtctccaggggaaagagccaccctctcctgcaggaccagtcagagtattggctggaacttagcctggtaccaacagaaacctggccaggctcccaggctcctcatctatggtgcatcttccaggaccactggtatcccagccaggttcagtggcagtgggtctgggacagagttcactctcaccatcagcagcctgcagtctgaagattctgcagtttattactgtcagcattatgataactggcccatgtgcagttttggccaggggaccgagctggagatcaaacgaactgtggctgcaccatctgtcttcatcttcccgccatctgatgagcagttgaaatctggaactgctagc

SEQ ID NO: 29 - protein, 128 aa, Homo sapiens
MRVPAQLLFLLLLWLPDTTGGIVMTQSPATLSVSPGERATLSCRTSQSIGWNLAWYQQKPGQAPRLLIYGASSRTTGIPARFSGSGSGTEFTLTISSLQSEDSAVYYCQHYDNWPMCSFGQGTELEIK

SEQ ID NO: 30 - DNA, 487 bp, Artificial Sequence
  Includes BamHI/Bg1II cloning junction, signal peptide, V region, portion of C region and 3XbaI/NheI (heavy) or NheI (light) cloning junction
ggatctcaccatggagtttgggctgtgctggattttcctcgttgctcttttaagaggtgtccagtgtcaggtgcagctggtggagtctgggggaggcgtggtccagcctgggaggtccctgagactctcctgtgcagcctctggattcaccttcattagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgagagtattagtgggagctttatattattataactactacgggatggacgtctggggccaagggaccacggtcaccgtctcctcagcctccaccaagggcccatcggtcttccccctggcaccctctagc

SEQ ID NO: 31 - protein, 144 aa, Homo sapiens
MEFGLCWIFLVALLRGVQCQVQLVESGGGVVQPGRSLRLSCAASGFTFISYGMHWVRQAPGKGLEWVAVISYDGSNKYYADSVKGRFTISRDNSKNTLYLQMNSLRAEDTAVYYCARVLVGALYYYNYYGMDVWGQGTTVTVSS

SEQ ID NO: 32 - DNA, 478 bp, Artificial Sequence
  Includes BamHI/Bg1II cloning junction, signal peptide, V region, portion of C region and 3XbaI/NheI (heavy) or NheI (light) cloning junction
ggatctcaccatgagggtccctgctcagctcctggggctgctaatgctctggatacctggatccagtgcagatattgtgatgacccagactccactctctctgtccgtcacccctggacagccggcctccatctcctgcaagtctagtcagagcctcctgcatagtgatggaaagacctttttgtattggtatctgcagaagccaggccagcctccacagctcctgatctatgaggtttccaaccggttctctggagtgccagataggttcagtggcagcgggtcagggacagatttcacactgaaaatcagccgggtggaggctgaggatgttgggctttattactgcatgcaaagtatacagcttccgctcactttcggcggagggaccaaggtggagatcaaacgaactgtggctgcaccatctgtcttcatcttcccgccatctgatgagcagttgaaatctggaactgctagc

SEQ ID NO: 33 - protein, 132 aa, Homo sapiens
MRVPAQLLGLLMLWIPGSSADIVMTQTPLSLSVTPGQPASISCKSSQSLLHSDGKTFLYWYLQKPGQPPQLLIYEVSNRFSGVPDRFSGSGSGTDFTLKISRVEAEDVGLYYCMQSIQLPLTFGGGTKVEIK